(19) United States
(12) Patent Application Publication
(10) Pub. No.: US 2023/0059371 A1
(43) Pub. Date: Feb. 23, 2023

(54) SUPPORT-GUIDED STEERING OF A CATHETER

(71) Applicant: CARDIOVALVE LTD., Or Yehuda (IL)

(72) Inventors: Ilia HARITON, Zichron Yaakov (IL); Oren SHUA, Elishama (IL); Meni IAMBERGER, Kfar Saba (IL)

(73) Assignee: CARDIOVALVE LTD., Or Yehuda (IL)

(21) Appl. No.: 17/797,289

(22) PCT Filed: Feb. 4, 2021
(86) PCT No.: PCT/IL2021/050132

§ 371 Date: Aug. 3, 2022

Related U.S. Application Data
(60) Provisional application No. 62/969,795, filed on Feb. 4, 2020.

(51) Int. Cl. A61B 17/00; A61M 25/01

(52) U.S. Cl. CPC A61B 17/00234; A61M 25/0102; A61M 25/0127; A61B 2017/00243; A61B 2017/00292

(57) ABSTRACT

Apparatus is described that includes a kit. The kit includes a flexible catheter (22) and a support (40). The catheter is transfemorally advanceable to a heart of a subject and the support is dimensioned for percutaneous access to the heart. The support has a proximal end, an elongate portion, and a distal portion (41) having a catheter-engaging element (38). The catheter-engaging element is configured to reversibly engage the catheter in a manner that does not inhibit longitudinal advancement of the catheter. Other embodiments are also described.

### CROSS-REFERENCES TO RELATED APPLICATIONS

This patent application claims benefit of U.S. Provisional application 62/969,795, filed Feb. 4, 2020, and entitled “SUPPORT-GUIDED STEERING,” which is incorporated herein by reference.

### FIELD OF THE INVENTION

Some applications of the present invention relate in general to cardiovascular medical procedures. Specifically, some applications of the present invention relate to use of a support to facilitate accessing native heart valves.

### BACKGROUND

Transluminal delivery is used in a range of cardiac medical procedures. Targets of such procedures include native heart valves. Typically, transluminally accessing a native heart valve involves steering a tool within the confines of the heart.

Transluminally accessing the tricuspid valve presents clinical challenges, including catheter navigation within the right atrium.

Transseptal delivery typically involves accessing the right atrium through the vasculature, followed by puncture of the atrial septum from within the right atrium. The fossa ovalis is considered an optimal location for transseptal puncture, as tissue of the fossa ovalis is typically thinner than other tissue of the septum.

Strain placed upon the septum during transseptal puncture and/or manipulation of tools across the septum, may result in stretching or tearing of septal tissue, contributing to the risk of postoperative complications.

### SUMMARY OF THE INVENTION

In accordance with some applications of the present invention, applying a supporting force to a portion of the delivery tool aids in navigation of a delivery tool while transluminally (e.g., transfemorally) navigating the delivery tool to an atrio-ventricular valve of the heart. For some applications, the supporting force is applied to support a supported portion of the delivery tool, which may serve as a pivot point with which the supporting force and a pushing force applied to the delivery tool can interact. Different intracatheter and extracatheter supports, and use of the supports in applying the supporting force to a delivery tool, are described herein.

For some applications, aspects of the present invention include implanting a prosthetic valve at a native valve of the heart. For some such applications, using the support to apply the supporting force to the delivery tool may facilitate coupling the prosthetic valve to ventricular tissue of the heart.

For some applications, a support is used to facilitate accessing a native heart valve from the right atrium. For example, a site of interest may be within the right side of the heart (e.g., the tricuspid valve), or may be within the left side of the heart (e.g., the mitral valve) but accessed transseptally from the right atrium. Typically for such applications, a catheter is advanced, via an inferior vena cava of the subject, to a right atrium of the heart, and a support is advanced, via a superior vena cava of the subject, to the right atrium.

For some applications, aspects of the present invention include engaging the delivery tool with the support within the right atrium of the heart, and steering a distal portion of the delivery tool toward a native valve, while using the support to support the delivery tool within the right atrium.

For some applications, aspects of the present invention include using an intracatheter support (e.g., comprising a stylet and/or a pull-wire) to apply the supporting force to the delivery tool. For example, the intracatheter support may be used to apply tension to a portion of the delivery tool.

For some applications, aspects of the present invention include engaging the catheter with a catheter-engaging element of an extracatheter support.

For some such applications, the catheter is mechanically engaged using the catheter-engaging element. For example, the distal portion may be advanced through a loop formed by the catheter-engaging element. Alternatively, a snare formed by the catheter-engaging element may be used to ensnare the catheter. For example, the catheter-engaging element may ensnare the catheter by transitioning the snare from a non-engaging state to an engaging state.

For some applications, the catheter is magnetically engaged with the catheter-engaging element. For example, an electromagnet may be activated to magnetically engage the catheter with the catheter-engaging element.

For some applications, the support is used to facilitate transseptally accessing a left atrium of a heart.

For some such applications, aspects of the present invention include advancing the catheter through an interatrial septum of the heart and into the left atrium. For example, the support may be used to support the catheter within the right atrium while the distal portion of the catheter is steered toward a mitral valve of the heart.

For example, the support may be used to support the catheter while implanting an implant (e.g., a prosthetic valve) at the native mitral valve.

For example, the support may be used to move the catheter by applying a force to the catheter. For some such applications, the force is applied to the catheter while the catheter is steered toward the mitral valve.

There is therefore provided, in accordance with an application of the present invention, an apparatus including a kit, the kit including:

a flexible catheter, the catheter being transfemorally advanceable to a heart of a subject; and

a support, the support being dimensioned for percutaneous access to the heart, the support defining:
a proximal end,
an elongate portion, and
a distal portion having a catheter-engaging element, the catheter-engaging element being configured to reversibly engage the catheter in a manner that does not inhibit longitudinal advancement of the catheter.

In an application, the catheter and the support are configured such that, while the catheter-engaging element engages the catheter, pulling on the proximal end of the support applies a superiorly-directed force to the catheter.

In an application, the catheter-engaging element is shaped to form a loop, the loop being dimensioned to facilitate advancing the catheter through the loop.

In an application, the loop is tightenable around the catheter when the loop is engaged with the catheter.

In an application, the apparatus includes a tightening ring, the loop being threaded through the tightening ring, such that advancing the tightening ring, along the loop, tightens the loop around the catheter.

In an application, the catheter-engaging element is transitionable in shape from a non-engaging state into an engaging state.

In an application, the apparatus includes a pull-wire attached to the catheter-engaging element, and the catheter-engaging element is configured to be bent, using the pull-wire, from the non-engaging state into the engaging state.

In an application, the apparatus includes a constraint, and the catheter-engaging element includes a shape-memory material, such that:

the catheter-engaging element is constrainable in the non-engaging state by the constraint, and upon removal of the constraint, the catheter-engaging element automatically transitions into the engaging state.

In an application, the constraint includes a constraining overtube, and the catheter-engaging element is disposed within the constraining overtube.

In an application, the constraint includes a stiff internal rod disposed within the catheter-engaging element.

In an application, the catheter-engaging element includes a snare, the snare configured to engage the catheter through an opening of the snare.

In an application, the opening of the snare has a width that is greater than an external diameter of the catheter.

In an application: the catheter includes a support-engaging element coupled to an outer wall of the catheter, and the catheter-engaging element is configured to engage the support-engaging element.

In an application: the support-engaging element is shaped to define a rail extending along a portion of the catheter,

the catheter-engaging element is shaped to define a hook, and

the hook is configured to ensnare the rail.

In an application:

the support-engaging element includes a ferromagnetic material,

the catheter-engaging element includes a ferromagnetic material, and

the catheter-engaging element is configured to magnetically engage the support-engaging element.

In an application, the support-engaging element includes an electromagnet.

In an application, the catheter-engaging element includes an electromagnet.

There is further provided, in accordance with an application of the present invention, an apparatus including a kit, the kit including:

a flexible catheter, the catheter:
being transfemorally advanceable to a heart of a subject, and
defining a primary lumen and a secondary lumen therethrough;

a prosthetic valve, the prosthetic valve being dimensioned to be passed to the heart through the primary lumen; and

a stylet that is stiffer than the flexible catheter, and is slidable through the secondary lumen.

In an application, the stylet is rigid.

In an application, the kit includes a piercing device, the piercing device being configured to pierce an interatrial septum of the heart.

In an application, the kit includes a dilator, the dilator being configured to dilate a pierced interatrial septum of the heart.

There is further provided, in accordance with an application of the present invention, a method for use at a heart of a subject, the method including:

advancing a distal end of a catheter transfemorally and through an interatrial septum of the heart into a left side of the heart; and

while a distal end of a stylet is disposed inside the catheter and within a right atrium of the heart, withdrawing the distal end of the catheter proximally toward the interatrial septum such that the catheter slides over the stylet.

In an application, the method includes, subsequently to the step of advancing the distal end of the catheter, and prior to the step of withdrawing the distal end of the catheter, advancing the distal end of the stylet through the catheter such that the distal end of the catheter becomes disposed within the right atrium of the heart, inside the catheter.

In an application, the step of advancing the distal end of the catheter includes advancing the distal end of the catheter transfemorally and through the interatrial septum of the heart into the left side of the heart while the distal end of the stylet is disposed inside the catheter.

In an application, the method includes, subsequently to withdrawing the distal end of the catheter proximally toward the interatrial septum, withdrawing the distal end of the catheter from the heart.

In an application, a prosthetic valve is coupled to the catheter, and the step of advancing the distal end of the catheter includes advancing the distal end of the catheter transfemorally and through the interatrial septum of the heart into the left side of the heart such that the prosthetic valve becomes advanced transfemorally and through the interatrial septum of the heart into the left side of the heart.

In an application, the method includes implanting the prosthetic valve at a mitral valve of the heart.

There is further provided, in accordance with an application of the present invention, a method for use at a heart of a subject, the method including:

advancing a distal portion of a catheter, via an inferior vena cava of the subject, to a right atrium of the heart, through an interatrial septum of the heart and into a left atrium of the heart;

advancing a support, via a superior vena cava of the subject, to the right atrium of the heart;

engaging the catheter with the support within the right atrium of the heart; and

while supporting the catheter in the right atrium using the support, withdrawing the distal end of the catheter proximally toward the interatrial septum.

There is further provided, in accordance with an application of the present invention, a method for transseptally accessing a left atrium of a heart of a subject, the method including:

advancing a catheter, via an inferior vena cava of the subject, to a right atrium of the heart;

advancing a support, via a superior vena cava of the subject, to the right atrium of the heart;

advancing a distal portion of the catheter through an interatrial septum of the heart and into the left atrium;

engaging the catheter with the support within the right atrium of the heart; and

subsequently, steering the distal portion of the catheter toward a mitral valve of the heart, while supporting the catheter in the right atrium using the support.

In an application, advancing the catheter to the right atrium includes advancing the catheter to the right atrium subsequently to advancing the support to the right atrium.

In an application, advancing the catheter to the right atrium includes advancing the catheter to the right atrium prior to advancing the support to the right atrium of the heart.

In an application, the support includes a catheter-engaging element, and engaging the catheter with the support includes engaging the catheter with the catheter-engaging element.

In an application, the catheter includes a support-engaging element, and engaging the catheter with the catheter-engaging element includes engaging the support-engaging element to the catheter-engaging element.

In an application, engaging the support-engaging element to the catheter-engaging element includes magnetically engaging the support-engaging element to the catheter-engaging element.

In an application, the support-engaging element includes an electromagnet, and magnetically engaging the support-engaging element to the catheter-engaging element includes activating the electromagnet.

In an application, the catheter-engaging element includes an electromagnet, and magnetically engaging the support-engaging element to the catheter-engaging element includes activating the electromagnet.

In an application:

the support-engaging element is a first support-engaging element,

magnetically engaging the support-engaging element to the catheter-engaging element includes magnetically engaging the first support-engaging element to the catheter-engaging element,

the catheter includes at least a second support-engaging element, and

the method includes, subsequently to magnetically engaging the first support-engaging element to the catheter-engaging element:
ceasing the magnetic engagement between the first support-engaging element and the catheter-engaging element by deactivating the electromagnet; and
subsequently, magnetically engaging the second support-engaging element to the catheter-engaging element by reactivating the electromagnet.

In an application, engaging the catheter with the catheter-engaging element includes mechanically engaging the catheter with the catheter-engaging element.

In an application, mechanically engaging the catheter with the catheter-engaging element includes fitting a protrusion into a recess.

In an application:

the catheter includes a support-engaging element shaped to define a rail,

the catheter-engaging element is shaped to define a hook, and

engaging the catheter with the catheter-engaging element includes hooking the rail with the hook.

In an application, the catheter-engaging element is shaped to form a loop, and mechanically engaging the catheter with the catheter-engaging element includes advancing the distal portion of the catheter through the loop of the catheter-engaging element.

In an application, the catheter-engaging element is shaped to define a snare, and mechanically engaging the catheter with the catheter-engaging element includes supporting a supported portion of the catheter with the snare.

In an application, supporting the supported portion includes supporting the supported portion while the supported portion is disposed within the right atrium.

In an application, the catheter-engaging element is shaped to form a loop, and supporting the supported portion with the catheter-engaging element includes tightening the loop around the catheter.

In an application, the catheter-engaging element has a non-engaging state and an engaging state, and advancing the support to the right atrium includes advancing the support while the catheter-engaging element is in the non-engaging state.

In an application, engaging the catheter includes transitioning the catheter-engaging element from the non-engaging state to the engaging state.

In an application, supporting the catheter in the right atrium using the support includes, subsequently to advancing the distal portion of the catheter through the interatrial septum, moving the catheter by applying a force to the catheter, using the support.

In an application, applying the force to the catheter includes applying a supporting force to the catheter using the support by applying tension to the support.

In an application, moving the catheter using the support includes using the support to change an orientation of the catheter.

In an application, supporting the catheter includes, subsequently to moving the catheter by applying the force to the catheter, continuing to apply the force to the catheter.

In an application, applying the force to the support facilitates steering the distal portion of the catheter toward the mitral valve.

In an application, moving the catheter includes moving a supported portion of the catheter, relative to the interatrial septum.

In an application, moving the supported portion of the catheter relative to the interatrial septum includes moving the supported portion of the catheter towards the superior vena cava.

In an application, the distal portion of the catheter includes a piercing device, and advancing the distal portion of the catheter through the interatrial septum includes piercing the septum with the piercing device.

In an application, the piercing device includes a needle, and piercing the septum includes mechanically puncturing the septum with the needle.

In an application:

advancing the catheter, via the inferior vena cava, to the right atrium includes advancing an implant within the catheter, via the inferior vena cava, to the right atrium; and

the method includes implanting the implant at the mitral valve.

In an application, the implant includes a prosthetic heart valve, and implanting the implant at the mitral valve includes implanting the prosthetic heart valve at the mitral valve.

In an application, the implant includes an annuloplasty device, and implanting the implant at the mitral valve includes implanting the annuloplasty device at the mitral valve.

There is further provided, in accordance with an application of the present invention, a method for transfemorally implanting a prosthetic valve at a native valve of a heart of a subject, the method including:

advancing a delivery tool, via an inferior vena cava of the subject, to a right atrium of the heart;

advancing a support, via a superior vena cava of the subject, to the right atrium of the heart;

engaging the delivery tool with the support within the right atrium of the heart;

steering a distal portion of the delivery tool toward the native valve, while supporting the delivery tool in the right atrium using the support; and

subsequently, at the native valve, deploying a prosthetic valve from the delivery tool.

In an application, the support includes a catheter-engaging element having a non-engaging state and the engaging state, and engaging the delivery tool with the support within the right atrium of the heart includes transitioning the catheter-engaging element from the non-engaging state to the engaging state.

In an application:

steering the distal portion of the delivery tool toward the native valve includes steering the distal portion of the delivery tool toward a tricuspid valve of the heart; and

deploying the prosthetic valve from the delivery tool includes deploying the prosthetic valve from the delivery tool at the tricuspid valve.

In an application, the method includes subsequently to deploying the prosthetic valve from the delivery tool:

withdrawing the support from the right atrium, via the superior vena cava; and

withdrawing the delivery tool from the right atrium, via the inferior vena cava.

In an application:

the delivery tool includes a proximal housing and a distal housing,

deploying the prosthetic valve from the delivery tool includes increasing a distance between the proximal housing and the distal housing, and

withdrawing the delivery tool proximally via the inferior vena cava includes retracting the distal housing through the prosthetic valve.

In an application, the method includes, subsequently to deploying the prosthetic valve from the delivery tool, disengaging the delivery tool from the support.

In an application, the support includes a catheter-engaging element having a non-engaging state and the engaging state, and disengaging the delivery tool from the support includes transitioning the catheter-engaging element from the engaging state to the non-engaging state.

In an application, supporting the delivery tool in the right atrium using the support includes applying a force to the delivery tool using the support.

In an application, applying the force to the delivery tool using the support includes moving the delivery tool using the support, by applying the force.

In an application, moving the delivery tool includes changing an orientation of the distal portion of the delivery tool.

In an application, applying the force to the delivery tool using the support includes applying the force to the delivery tool using the support while the prosthetic valve is at least partially disposed within a right ventricle of the heart.

In an application, applying the force to the delivery tool using the support includes applying the force to the delivery tool using the support while the prosthetic valve is at least partially deployed from the delivery tool.

In an application, applying the force to the delivery tool using the support includes coupling the prosthetic valve to a ventricular tissue of the heart.

In an application, applying the force to the delivery tool using the support includes applying a superiorly-directed force to the delivery tool using the support.

In an application, applying the superiorly-directed force to the delivery tool using the support includes applying the superiorly-directed force to the delivery tool using the support, while deploying the prosthetic valve from the delivery tool.

In an application, applying the superiorly-directed force to the delivery tool using the support includes, subsequently to deploying the prosthetic valve, applying the superiorly-directed force to the delivery tool using the support while withdrawing at least a portion of the delivery tool into the right atrium.

In an application, applying the superiorly-directed force to the delivery tool using the support while withdrawing at least the portion of the delivery tool into the right atrium includes applying the superiorly-directed force to the delivery tool using the support while withdrawing the distal housing into the right atrium.

There is further provided, in accordance with an application of the present invention, a method for transseptally accessing a left atrium of a heart of a subject, the method including:

transluminally positioning a delivery tool including a catheter and a stylet such that:
the catheter extends, via an inferior vena cava of the subject, to a right atrium of the heart, and
the stylet extends, within the catheter, to the right atrium; and while a distal end of the stylet remains in the right atrium:
steering a distal portion of the catheter away from the distal end of the stylet and through an interatrial septum of the heart into the left atrium; and
subsequently, steering the distal portion of the catheter toward a mitral valve of the heart.

In an application, extending the catheter, via the inferior vena cava, to the right atrium, includes extending the catheter, via the inferior vena cava, to the right atrium, while the catheter is empty of the stylet.

In an application, extending the catheter, via the inferior vena cava, to the right atrium, includes extending the catheter, via the inferior vena cava, to the right atrium, while the stylet is disposed within the catheter.

In an application, the catheter defines a secondary lumen therethrough, and extending the stylet within the catheter and to the right atrium includes extending the stylet through the secondary lumen.

In an application, the catheter houses an implant, and the method includes:

advancing the implant within the catheter, via the inferior vena cava, to the right atrium; and

subsequently to steering the distal portion of the catheter toward the mitral valve, deploying the implant from within catheter, at the mitral valve.

In an application, the implant includes a prosthetic heart valve, and implanting the implant at the mitral valve includes implanting the prosthetic heart valve at the mitral valve.

In an application, the implant includes an annuloplasty device, and implanting the implant at the mitral valve includes implanting the annuloplasty device at the mitral valve.

In an application, the delivery tool further includes a piercing device, the piercing device extending distally out of the distal end of catheter, and steering the distal end of the catheter through the interatrial septum includes piercing the septum with the piercing device.

In an application, the piercing device includes a needle, and piercing the septum includes mechanically puncturing the septum with the needle.

In an application, steering the distal portion of the catheter away from the distal end of the stylet and through the interatrial septum includes steering the distal portion of the catheter away from the distal end of the stylet and through the interatrial septum while the stylet extends within the right atrium, to an atrial height that is between 1 and 70 mm.

In an application, steering the distal portion of the catheter away from the distal end of the stylet and through the interatrial septum while the stylet extends within the right atrium, to an atrial height that is between 1 and 70 mm includes steering the distal portion of the catheter away from the distal end of the stylet and through the interatrial septum while the stylet extends within the right atrium, to an atrial height that is between 1 and 10 mm.

In an application, steering the distal portion of the catheter away from the distal end of the stylet and through the interatrial septum while the stylet extends within the right atrium, to an atrial height that is between 1 and 70 mm includes steering the distal portion of the catheter away from the distal end of the stylet and through the interatrial septum while the stylet extends within the right atrium, to an atrial height that is between 10 and 20 mm.

In an application, steering the distal portion of the catheter away from the distal end of the stylet and through the interatrial septum while the stylet extends within the right atrium, to an atrial height that is between 1 and 70 mm includes steering the distal portion of the catheter away from the distal end of the stylet and through the interatrial septum while the stylet extends within the right atrium, to an atrial height that is between 10 and 70 mm.

In an application, steering the distal portion of the catheter away from the distal end of the stylet and through the interatrial septum while the stylet extends within the right atrium, to an atrial height that is between 10 and 70 mm includes steering the distal portion of the catheter away from the distal end of the stylet and through the interatrial septum while the stylet extends within the right atrium, to an atrial height that is between 20 and 70 mm.

In an application, steering the distal portion of the catheter away from the distal end of the stylet and through the interatrial septum while the stylet extends within the right atrium, to an atrial height that is between 20 and 70 mm includes steering the distal portion of the catheter away from the distal end of the stylet and through the interatrial septum while the stylet extends within the right atrium, to an atrial height that is between 20 and 30 mm.

In an application, steering the distal portion of the catheter away from the distal end of the stylet and through the interatrial septum while the stylet extends within the right atrium, to an atrial height that is between 20 and 70 mm includes steering the distal portion of the catheter away from the distal end of the stylet and through the interatrial septum while the stylet extends within the right atrium, to an atrial height that is between 30 and 60 mm.

In an application, steering the distal portion of the catheter away from the distal end of the stylet and through the interatrial septum while the stylet extends within the right atrium, to an atrial height that is between 20 and 70 mm includes steering the distal portion of the catheter away from the distal end of the stylet and through the interatrial septum while the stylet extends within the right atrium, to an atrial height that is between 40 and 70 mm.

In an application, steering the distal portion of the catheter toward the mitral valve, while the distal end of the stylet remains in the right atrium, includes steering the distal portion of the catheter toward the mitral valve, while the stylet extends within the right atrium, to an atrial height that is between 1 and 70 mm.

In an application, steering the distal portion of the catheter toward the mitral valve, while the stylet extends within the right atrium, to an atrial height that is between 1 and 70 mm, includes steering the distal portion of the catheter toward the mitral valve, while the stylet extends within the right atrium, to an atrial height that is between 1 and 10 mm.

In an application, steering the distal portion of the catheter toward the mitral valve, while the stylet extends within the right atrium, to an atrial height that is between 1 and 70 mm, includes steering the distal portion of the catheter toward the mitral valve, while the stylet extends within the right atrium, to an atrial height that is between 10 and 20 mm.

In an application, steering the distal portion of the catheter toward the mitral valve, while the stylet extends within the right atrium, to an atrial height that is between 1 and 70 mm, includes steering the distal portion of the catheter toward the mitral valve, while the stylet extends within the right atrium, to an atrial height that is between 10 and 70 mm.

In an application, steering the distal portion of the catheter toward the mitral valve, while the stylet extends within the right atrium, to an atrial height that is between 10 and 70 mm, includes steering the distal portion of the catheter toward the mitral valve, while the stylet extends within the right atrium, to an atrial height that is between 20 and 70 mm.

In an application, steering the distal portion of the catheter toward the mitral valve, while the stylet extends within the right atrium, to an atrial height that is between 20 and 70 mm, includes steering the distal portion of the catheter toward the mitral valve, while the stylet extends within the right atrium, to an atrial height that is between 20 and 30 mm.

In an application, steering the distal portion of the catheter toward the mitral valve, while the stylet extends within the right atrium, to an atrial height that is between 20 and 70 mm, includes steering the distal portion of the catheter toward the mitral valve, while the stylet extends within the right atrium, to an atrial height that is between 30 and 60 mm.

In an application, steering the distal portion of the catheter toward the mitral valve, while the stylet extends within the right atrium, to an atrial height that is between 20 and 70 mm, includes steering the distal portion of the catheter toward the mitral valve, while the stylet extends within the right atrium, to an atrial height that is between 40 and 70 mm.

There is further provided, in accordance with an application of the present invention, a method for transfemorally accessing a native valve of a heart of a subj ect, the method including:

positioning a delivery tool including a catheter and a stylet, such that:
the catheter extends, via an inferior vena cava of the subject, to a right atrium of the heart, and
the stylet extends, within the catheter, to the right atrium; and while a distal end of the stylet remains in the right atrium, and within the catheter:
steering a distal portion of the delivery tool away from the distal end of the stylet and toward the native valve.

In an application, the method includes, subsequently to the step of steering:

deploying a prosthetic valve from the delivery tool, at the native valve.

In an application, the native valve is a tricuspid valve of the heart.

In an application:

the native valve is a mitral valve of the heart, and

the step of steering includes advancing the distal portion of the delivery tool through an interatrial septum of the heart into a left atrium of the heart.

In an application, the catheter defines a secondary lumen therethrough, and extending the stylet within the catheter and to the right atrium includes extending the stylet through the secondary lumen.

In an application, positioning the delivery tool includes advancing the delivery tool transluminally via the inferior vena cava.

In an application, positioning the delivery tool includes positioning the delivery tool such that the stylet extends, within the catheter and through a secondary lumen, to the right atrium.

In an application, steering the distal portion of the delivery tool away from the distal end of the stylet and toward the native valve includes sliding the catheter distally over the stylet.

In an application, sliding the catheter distally over the stylet includes holding the distal end of the stylet at a generally constant atrial height while steering the distal portion toward and through the native valve.

In an application, deploying the prosthetic valve from the distal portion of the delivery tool includes extracorporeally applying a proximally-directed force to the delivery tool.

In an application, extracorporeally applying the proximally-directed force to the delivery tool includes extracorporeally applying the proximally-directed force to the delivery tool such that the prosthetic valve moves proximally and engages tissue of the native valve.

In an application, the delivery tool includes a distal housing, and extracorporeally applying the proximally-directed force to the delivery tool includes increasing a distance between a distal opening of the catheter and the distal housing.

In an application, extracorporeally applying the proximally-directed force to the delivery tool includes retracting the delivery tool through the prosthetic valve.

In an application, extracorporeally applying the proximally-directed force to the delivery tool includes holding the distal end of the stylet in the right atrium, and within the catheter, a generally constant atrial height, by applying the proximally-directed force to catheter, and applying a pushing force to the stylet.

In an application, steering the distal portion of the delivery tool away from the distal end of the stylet and toward the native valve, while the distal end of the stylet remains in the right atrium, and within the catheter, includes steering the distal portion of the catheter away from the distal end of the stylet and toward the native valve, while the stylet extends within the right atrium, to an atrial height that is between 1 and 70 mm.

In an application, steering the distal portion of the catheter away from the distal end of the stylet and toward the native valve, while the stylet extends within the right atrium, to an atrial height that is between 1 and 70 mm includes steering the distal portion of the catheter away from the distal end of the stylet and toward the native valve, while the stylet extends within the right atrium, to an atrial height that is between 1 and 10 mm.

In an application, steering the distal portion of the catheter away from the distal end of the stylet and toward the native valve, while the stylet extends within the right atrium, to an atrial height that is between 1 and 70 mm includes steering the distal portion of the catheter away from the distal end of the stylet and toward the native valve, while the stylet extends within the right atrium, to an atrial height that is between 10 and 20 mm.

In an application, steering the distal portion of the catheter away from the distal end of the stylet and toward the native valve, while the stylet extends within the right atrium, to an atrial height that is between 1 and 70 mm includes steering the distal portion of the catheter away from the distal end of the stylet and toward the native valve, while the stylet extends within the right atrium, to an atrial height that is between 10 and 70 mm.

In an application, steering the distal portion of the catheter away from the distal end of the stylet and toward the native valve, while the stylet extends within the right atrium, to an atrial height that is between 10 and 70 mm includes steering the distal portion of the catheter away from the distal end of the stylet and toward the native valve, while the stylet extends within the right atrium, to an atrial height that is between 20 and 70 mm.

In an application, steering the distal portion of the catheter away from the distal end of the stylet and toward the native valve, while the stylet extends within the right atrium, to an atrial height that is between 20 and 70 mm includes steering the distal portion of the catheter away from the distal end of the stylet and toward the native valve, while the stylet extends within the right atrium, to an atrial height that is between 20 and 30 mm.

In an application, steering the distal portion of the catheter away from the distal end of the stylet and toward the native valve, while the stylet extends within the right atrium, to an atrial height that is between 20 and 70 mm includes steering the distal portion of the catheter away from the distal end of the stylet and toward the native valve, while the stylet extends within the right atrium, to an atrial height that is between 30 and 60 mm.

In an application, steering the distal portion of the catheter away from the distal end of the stylet and toward the native valve, while the stylet extends within the right atrium, to an atrial height that is between 20 and 70 mm includes steering the distal portion of the catheter away from the distal end of the stylet and toward the native valve, while the stylet extends within the right atrium, to an atrial height that is between 40 and 70 mm.

In an application, deploying the prosthetic valve from the delivery tool, while the distal end of the stylet remains in the right atrium, and within the catheter, includes deploying the prosthetic valve from the delivery tool while the stylet extends within the right atrium, to an atrial height that is between 1 and 70 mm.

In an application, deploying the prosthetic valve from the delivery tool while the stylet extends within the right atrium, to an atrial height that is between 1 and 70 mm, includes deploying the prosthetic valve from the delivery tool while the stylet extends within the right atrium, to an atrial height that is between 1 and 10 mm.

In an application, deploying the prosthetic valve from the delivery tool while the stylet extends within the right atrium, to an atrial height that is between 1 and 70 mm, includes deploying the prosthetic valve from the delivery tool while the stylet extends within the right atrium, to an atrial height that is between 10 and 20 mm.

In an application, deploying the prosthetic valve from the delivery tool while the stylet extends within the right atrium, to an atrial height that is between 1 and 70 mm includes deploying the prosthetic valve from the delivery tool while the stylet extends within the right atrium, to an atrial height that is between 10 and 70 mm.

In an application, deploying the prosthetic valve from the delivery tool while the stylet extends within the right atrium, to an atrial height that is between 10 and 70 mm includes deploying the prosthetic valve from the delivery tool while the stylet extends within the right atrium, to an atrial height that is between 20 and 70 mm.

In an application, deploying the prosthetic valve from the delivery tool while the stylet extends within the right atrium, to an atrial height that is between 20 and 70 mm includes deploying the prosthetic valve from the delivery tool while the stylet extends within the right atrium, to an atrial height that is between 20 and 30 mm.

In an application, deploying the prosthetic valve from the delivery tool while the stylet extends within the right atrium, to an atrial height that is between 20 and 70 mm includes deploying the prosthetic valve from the delivery tool while the stylet extends within the right atrium, to an atrial height that is between 30 and 60 mm.

In an application, deploying the prosthetic valve from the delivery tool while the stylet extends within the right atrium, to an atrial height that is between 20 and 70 mm includes deploying the prosthetic valve from the delivery tool while the stylet extends within the right atrium, to an atrial height that is between 40 and 70 mm.

There is further provided, in accordance with an application of the present invention, a method for transseptally accessing a mitral valve of a heart of a subject, the method including:

transluminally positioning a catheter such that the catheter extends, via an inferior vena cava of the subject, to a right atrium of the heart;

advancing a distal portion of the catheter through an interatrial septum of the heart into a left atrium of the heart;

subsequently, tilting the distal portion of the catheter toward the mitral valve by transluminally extending a stylet, within the catheter, to the right atrium.

In an application, transluminally extending the stylet, within the catheter, to the right atrium, includes extracorporeally pushing a proximal portion of the stylet towards a superior vena cava of the heart.

In an application, tilting the distal portion of the catheter includes tilting the distal portion of the catheter such that the distal opening of the catheter faces the mitral valve.

In an application, while a distal end of the stylet remains in the right atrium, steering the distal portion of the catheter toward a mitral valve of the heart.

In an application, the catheter houses an implant, and the method includes, subsequently to tilting the distal portion of the catheter, deploying the implant from within the catheter, at the mitral valve.

In an application, the implant includes a prosthetic heart valve, and implanting the implant at the mitral valve includes implanting the prosthetic heart valve at the mitral valve.

In an application, the implant includes an annuloplasty device, and implanting the implant at the mitral valve includes implanting the annuloplasty device at the mitral valve.

In an application, transluminally extending the stylet, within the catheter, to the right atrium, includes extending the stylet, within the right atrium, to an atrial height between 1 and 70 mm.

In an application, extending the stylet, within the right atrium, to an atrial height between 1 and 70 mm, includes extending the stylet to an atrial height between 1 and 10 mm.

In an application, extending the stylet, within the right atrium, to an atrial height between 1 and 70 mm, includes extending the stylet to an atrial height between 10 and 20 mm.

In an application, the stylet, within the right atrium, to an atrial height between 1 and 70 mm, includes extending the stylet to an atrial height between 10 and 70 mm.

In an application, extending the stylet, within the right atrium, to an atrial height between 10 and 70 mm, includes extending the stylet to an atrial height between 20 and 70 mm.

In an application, extending the stylet to an atrial height between 20 and 70 mm, includes extending the stylet to an atrial height between 20 and 30 mm.

In an application, extending the stylet to an atrial height between 20 and 70 mm, includes extending the stylet to an atrial height between 30 and 60 mm.

In an application, extending the stylet to an atrial height between 20 and 70 mm, includes extending the stylet to an atrial height between 40 and 70 mm.

Other embodiments are also described.

The present invention will be more fully understood from the following detailed description of applications thereof, taken together with the drawings, in which:

### BRIEF DESCRIPTION OF THE DRAWINGS

FIGS. 1A-1F and 2 are schematic illustrations showing use of a multi-component system comprising a support and a delivery tool to transseptally access a left atrium of a heart of a subject, in accordance with some applications of the present invention;

FIGS. 3A-B are schematic illustrations showing use of another multi-component system comprising a support and the delivery tool, for transseptally accessing the left atrium, in accordance with some applications of the invention;

FIGS. 4A-C are schematic illustrations showing use of another multi-component system comprising a support and the delivery tool, for transseptally accessing the left atrium, in accordance with some applications of the invention;

FIGS. 5A-C are schematic illustrations showing use of another multi-component system comprising a support and delivery tool, for transseptally accessing the left atrium, in accordance with some applications of the invention;

FIGS. 6A-C are schematic illustrations showing use of another multi-component system comprising a support and a delivery tool, for transseptally accessing the left atrium, in accordance with some applications of the invention;

FIGS. 7A-C are schematic illustrations showing use of another multi-component system comprising a support and a delivery tool, for transseptally accessing the left atrium, in accordance with some applications of the invention;

FIGS. 8A-8L, and 9 are schematic illustrations showing use of another multi-component system comprising a support and a delivery tool, for transfemorally implanting a prosthetic valve at a native valve of the heart, in accordance with some applications of the present invention;

FIGS. 10A-F and 11A-B are schematic illustrations showing use of a delivery tool comprising a catheter and a stylet for transseptally accessing the left atrium, in accordance with some applications of the invention;

FIGS. 12A-J are schematic illustrations showing use of another delivery tool comprising a catheter and a stylet for transfemorally implanting a prosthetic valve at a native valve of the heart, in accordance with some applications of the present invention;

FIGS. 13A-B, and 14A-D are schematic illustrations showing transfemoral-transseptal access to the left atrium of a subject, either with or without facilitation with a stylet, in accordance with some applications of the invention; and

FIGS. 15-16 are schematic illustrations showing use of a delivery tool comprising a catheter and a pull-wire, for transfemorally implanting a prosthetic valve at a native valve of the heart, in accordance with some applications of the present invention.

### DETAILED DESCRIPTION OF EMBODIMENTS

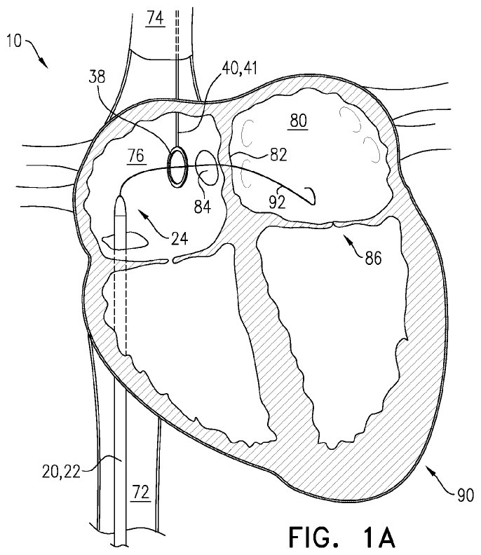

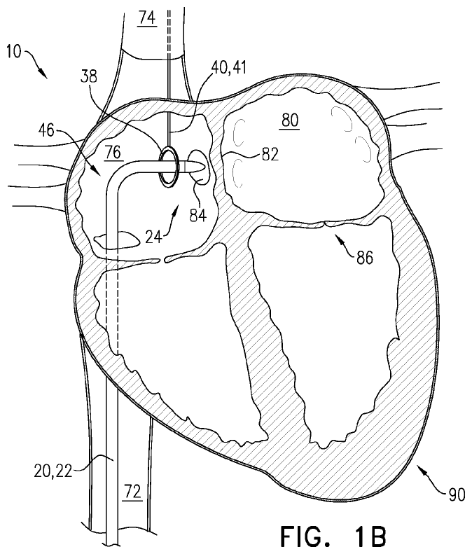

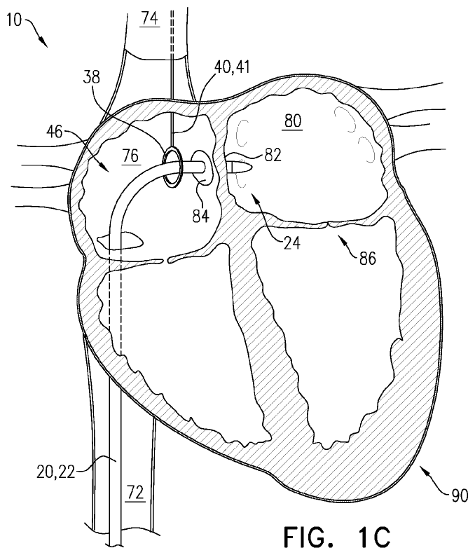

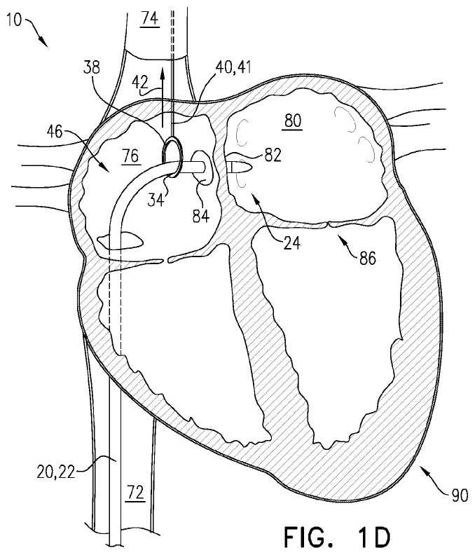

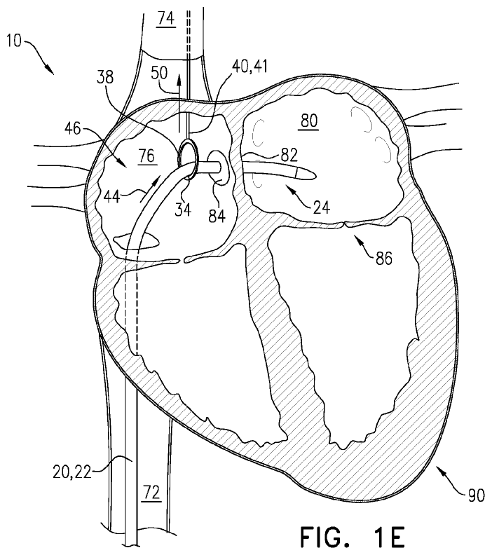

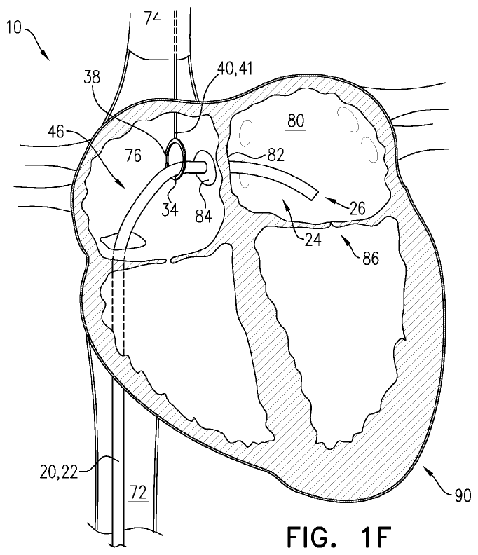

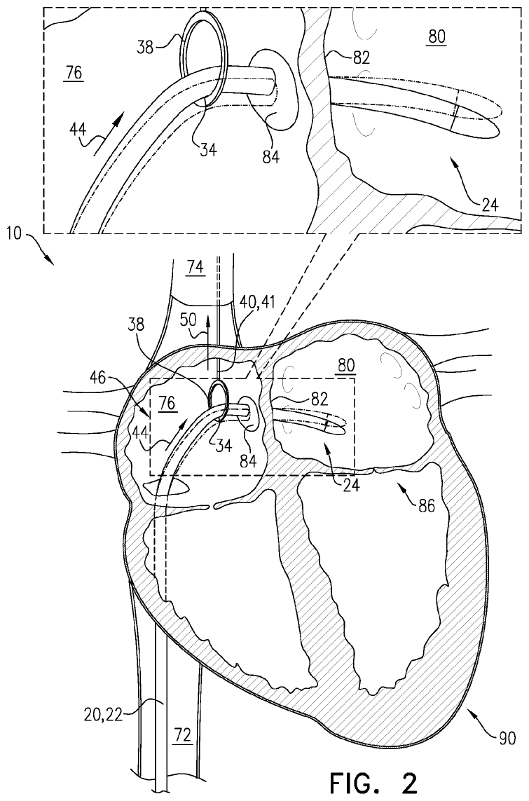

Reference is made to FIGS. 1A-1F and 2, which are schematic illustrations showing use of a multi-component system 10 comprising a support 40 and a delivery tool 20 to transseptally access a left atrium 80 of a heart 90 of a subject, in accordance with some applications of the present invention.

For some applications, elements of system 10 (e.g., delivery tool 20 and support 40) are packaged together commercially, e.g., in a kit.

As shown in FIG. 1A, a delivery tool 20 comprising a catheter 22 is advanced (in accordance with some applications, along a guidewire 92, shown in FIG. 1A but not in FIGS. 1B-1F and 2), via an inferior vena cava 72, such that a distal portion 24 of the catheter reaches a right atrium 76 of heart 90. Additionally, a support 40 (e.g., a distal portion 41 thereof) is advanced, via the superior vena cava 74 (e.g., reached through a jugular vein), such that a catheter-engaging element 38 of the support reaches right atrium 76 of the heart. For some applications, and as shown, catheter-engaging element 38 is shaped to form a loop (e.g., a closed loop).

Some other embodiments of catheter-engaging elements are described hereinbelow.

For some applications, distal portion 24 of catheter 22 is advanced to right atrium 76 prior to advancing catheter-engaging element 38 of support 40 to the right atrium. For other applications, distal portion 24 of catheter 22 is advanced to right atrium 76 subsequently to advancing catheter-engaging element 38 of support 40 to the right atrium. Alternatively, distal portion 24 and catheter-engaging element 38 may be advanced simultaneously to right atrium 76.

Typically, and as shown, catheter 22 is engaged with catheter-engaging element 38 while the catheter-engaging element is disposed within right atrium 76. As shown in FIG. 1B, catheter 22 is typically engaged with catheter-engaging element 38 while both the catheter-engaging element and distal portion 24 of the catheter are disposed within right atrium 76.

For some applications in which catheter-engaging element 38 is shaped to form a loop, and as shown, the catheter-engaging element is used to mechanically engage catheter 22 initially by threading distal portion 24 of the catheter through the catheter-engaging element (FIG. 1B). Typically, the loop has an area that is at least equal to a cross-section of the catheter.

Typically for such applications, distal portion 24 is threaded through catheter-engaging element 38 while the distal portion and the catheter-engaging element are both disposed within right atrium 76. For some applications wherein catheter-engaging element 38 is shaped to form a loop, distal portion 24 is threaded through catheter-engaging element 38 by advancing the distal portion of the catheter through the loop of the catheter-engaging element, e.g., after catheter-engaging element 38 is pre-positioned in front of fossa ovalis 84. Alternatively or in addition, catheter-engaging element 38 may be moved longitudinally with respect to distal portion 24, such that catheter 22 passes through the loop.

Threading distal portion 24 through the loop may be facilitated by one or more techniques for visualization of delivery tool 20, support 40 and/or tissue of heart 90, including but not limited to fluoroscopy, transesophageal echocardiography (TEE) and dye staining of tissue.

As shown in FIG. 1C, distal portion 24 of catheter 22 is advanced through an interatrial septum 82 (e.g., a fossa ovalis 84 thereof) of heart 90 and into left atrium 80. For some applications, distal portion 24 of catheter 22 comprises a piercing device and/or a dilator (not shown). For some such applications, distal portion 24 is advanced through septum 82 by piercing the septum (e.g., with the piercing device). For example, the piercing device may comprise a needle, and the needle is used to mechanically puncture the septum. Other methods of piercing septum 82 (e.g., radiofrequency ablation and/or ultrasound ablation) are also contemplated.

For some such applications, catheter-engaging element 38 is used to support catheter 22 (e.g., a supported portion 34 of the catheter) during further advancement and/or steering of the catheter. Supported portion 34 is not necessarily a discrete part or feature of catheter 22, but rather the supported portion may be defined as a portion of the catheter currently being engaged by catheter-engaging element 38. For some applications, the operator may not need to engage a precise target on catheter 22 with catheter-engaging element 38. It is hypothesized by the inventors that obviating a need to engage a precise target portion of catheter 22 facilitates the use of support 40 to engage the catheter.

For some applications in which catheter-engaging element 38 is shaped to form a loop, the engagement of catheter 22 by catheter-engaging element 38 is achieved by advancing the catheter through the loop (FIGS. 1B, 1C). For such applications, catheter 22 is typically supported by support 40. For example, and as shown in FIG. 1D, moving 42 support 40 closer to superior vena cava 74 results in the support supporting supported portion 34 of catheter 22. For some such applications, and as described hereinbelow, moving support 40 (e.g., to supported portion 34 thereof) may result in moving the catheter.

FIG. 1D-1F show supported portion 34 being supported using catheter-engaging element 38 of support 40 while distal portion 24 is disposed within left atrium 80 (e.g., while the distal portion is steered toward mitral valve 86).

For some applications, catheter 22 being supported using catheter-engaging element 38 enables application of a supporting force 50 (e.g., a superiorly-directed force) from support 40 to the catheter (FIG. 1E). Typically, the force is applied by the operator by applying tension to (e.g., pulling) support 40 (e.g., by pulling on a proximal end of the support, disposed outside of the body of the subject). For some such applications, and as shown in FIG. 1E, the application of force from support 40 to catheter 22 moves the catheter, e.g., supported portion 34 thereof, e.g., as indicated by arrow 44. For some such applications, and as shown, catheter 22 is moved subsequently to advancing distal portion 24 of the catheter through interatrial septum 82 (e.g., while the distal portion is disposed within left atrium 80). Alternatively, catheter 22 is moved while distal portion 24 is disposed within right atrium 76.

For some applications, alternatively or in addition to moving supported portion 34, applying force 50 to supported portion 34 changes an orientation of the catheter, e.g., an orientation of a right atrial portion 46 thereof, e.g., as shown by the transition between FIGS. 1D and 1E. Right atrial portion 46 is typically not defined by a discrete feature of catheter 22. Instead, right atrial portion 46 is typically defined by virtue of this portion of catheter 22 being disposed within right atrium 76. That is, successive portions of catheter 22 may be referred to as right atrial portion 46, as the catheter is advanced through the right atrium.

Typically, the force is continually applied to supported portion 34 while distal portion 24 is steered to mitral valve 86, as shown in FIG. 1F. For some applications the currently supported portion 34 therefore changes during advancement of the catheter. In this way, catheter-engaging element 38 may support progressively proximal supported portions 34 as the catheter is advanced and/or steered toward mitral valve 86.

For some applications, although the superiorly-directed force applied to catheter using support 40 allows longitudinal advancement of the catheter (e.g., orthogonal to the plane of septum 82), the force typically nonetheless restricts lateral movement, in at least one direction, of supported portion 34 while distal portion 24 of catheter 22 is steered toward mitral valve 86. It is hypothesized by the inventors that restricting lateral movement of the supported portion reduces shearing forces applied by catheter 22 upon tissue of interatrial septum 82 while distal portion 24 of catheter 22 is advanced and/or steered toward mitral valve 86, thereby reducing a risk of stretching or tearing (e.g., “cheesewiring”) tissue of septum 82. For some applications, supported portion 34 may serve as a fulcrum about which portions of catheter 22 may pivot in response to forces applied to the catheter.

For some applications, the operator determines a strength of the force applied using support 40 to supported portion 34, thereby determining a degree to which the catheter (e.g., the supported portion and/or right atrial portion 46 thereof) is moved. For example, FIG. 2 shows effects of application of forces of varying strengths upon the orientation of catheter 22. Solid lines of catheter 22 depict the orientation of catheter 22 of application, using support 40, of a stronger force than that applied in FIGS. 1E-F. As shown, supported portion 34 is pulled more strongly towards superior vena cava 74 than in FIG. 1D. The effect of the stronger force upon the orientation of catheter 22 is illustrated relative to the orientation of the catheter shown in FIGS. 1E-F, which are shown in phantom in FIG. 2. It is hypothesized by the inventors that applying force (e.g., applying the stronger force to more sharply change the orientation of right atrial portion 46) facilitates steering distal portion 24 of catheter 22 toward mitral valve 86, e.g., by changing the angle at which the catheter passes through fossa ovalis 84.

Typically, accessing left atrium 80 of heart 90 using delivery tool 20 facilitates performance of one or more clinical interventions, including but not limited to mitral annuloplasty, mitral chord repair, mitral valve replacement, left atrial appendage occlusion, or ablation for atrial fibrillation.

Some embodiments of the current invention are used to transseptally implant an implant to mitral valve 86 of heart 90. For example, the implant may be a prosthetic heart valve or an annuloplasty device. Typically for such applications, the implant is advanced within catheter 22, via inferior vena cava 72, to right atrium 76. Further typically, support 40 is used to support catheter 22 in right atrium 76, as described hereinabove, while distal portion 24 of catheter 22 is steered toward mitral valve 86, and while the implant is implanted at the mitral valve.

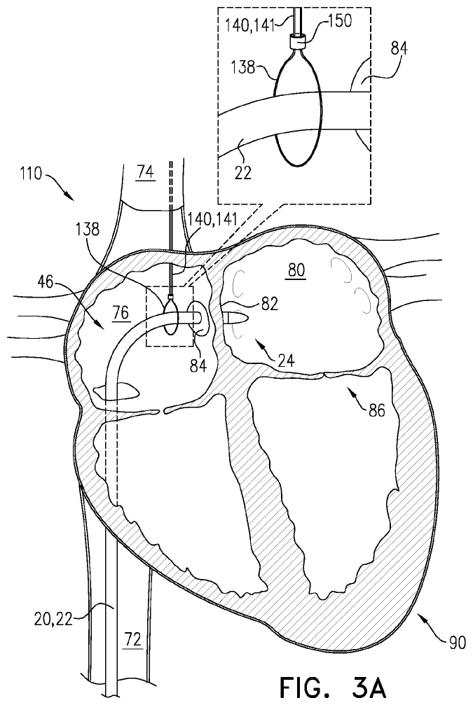

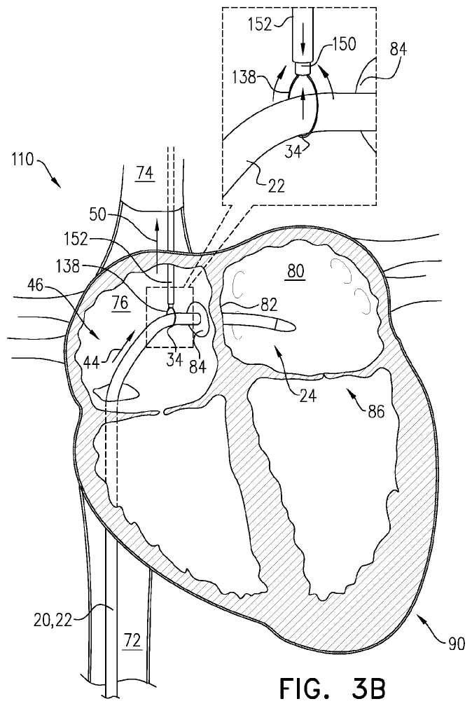

Reference is made to FIGS. 3A-B, which are schematic illustrations showing use of a multi-component system 120 comprising a support 140 and delivery tool 20, for transseptally accessing left atrium 80 of heart 90 of a subject, in accordance with some applications of the invention.

For some applications, elements of system 120 (e.g., delivery tool 20 and support 140) are packaged together commercially, e.g., in a kit.

Except where noted, system 120 is typically identical to system 10 described hereinabove, and is used similarly as system 10, mutatis mutandis. Components that are identically named between the systems typically share similar features and serve similar functions as each other. For example, system 120 comprises delivery tool 20, similarly to as described hereinabove in reference to FIGS. 1A-1F, mutatis mutandis. As such, the description below of system 120 focuses upon features that are particular to system 120 (e.g., the use of a support 140).

FIG. 3A shows a distal portion 141 of support 140, comprising a catheter-engaging element 138, having been advanced to right atrium 76. Similarly to catheter-engaging element 38 described hereinabove, catheter-engaging element 138 of support 140 is shaped to form a loop. Further similarly to catheter-engaging element 38, catheter 22 is advanced (e.g., threaded) through catheter-engaging element 138, such that the catheter-engaging element engages the catheter.

In contrast to catheter-engaging element 38 described above, the loop formed by catheter-engaging element 138 may be tightened around catheter 22 (e.g., supported portion 34 thereof), such that the catheter-engaging element supports the catheter. For some applications, and as shown in inset of FIG. 3A, catheter-engaging element 138 further comprises a tightening ring 150 through which catheter-engaging element 138 is threaded. For some such applications, and as shown, the loop formed by catheter-engaging element 138 is tightened around catheter 22 by advancing a tightening tube 152 toward tightening ring 150 (FIG. 3B), such that a circumference of the loop formed by the catheter-engaging element is reduced. For some applications, tightening the loop formed by catheter-engaging element 138 around catheter 22 may further restrict forces nonorthogonal to fossa ovalis 84 (e.g., lateral movement of the catheter) while steering distal portion 24 of catheter 22 toward mitral valve 86, further reducing a risk of stretching or tearing tissue of septum 82.

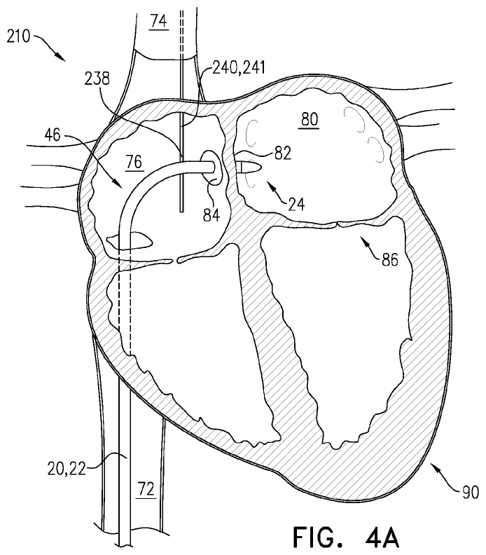

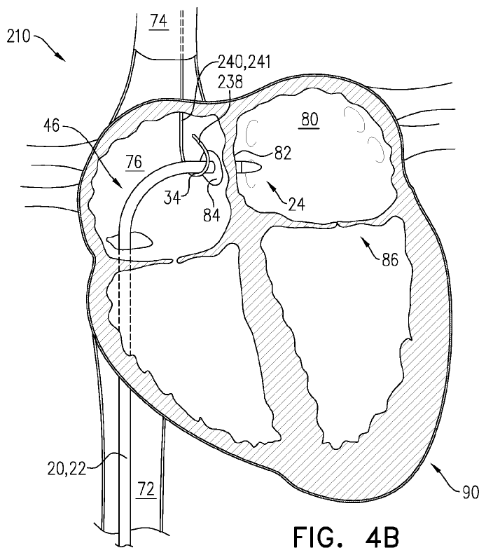

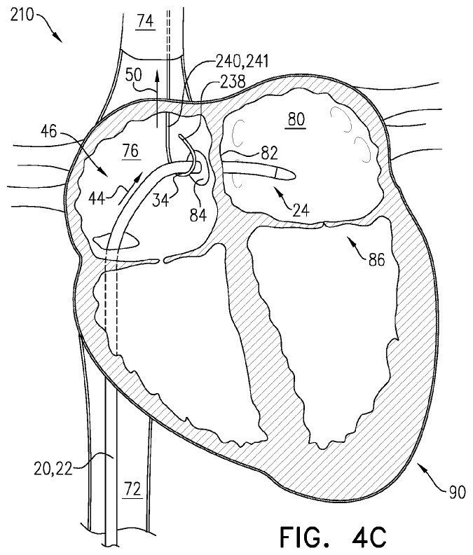

Reference is made to FIGS. 4A-C, which are schematic illustrations showing use of a multi-component system 210 comprising a support 240 and delivery tool 20 in transseptally accessing left atrium 80 of heart 90 of a subject, in accordance with some applications of the invention.

For some applications, elements of system 210 (e.g., delivery tool 20 and support 240) are packaged together commercially, e.g., in a kit.

Except where noted, system 210 is typically identical to system 10 described hereinabove, and is used similarly as system 10, mutatis mutandis. Components that are identically named between the systems typically share similar features and serve similar functions as each other. For example, system 210 comprises delivery tool 20, similarly to as described hereinabove in reference to FIGS. 1A-1F, mutatis mutandis. As such, the description below of system 210 focuses upon features that are particular to system 210 (e.g., the use of a support 240).

FIG. 4A shows a distal portion 241 of support 240, comprising a catheter-engaging element 238, having been advanced to right atrium 76. In contrast to other catheter-engaging elements described hereinabove, catheter-engaging element 238 has a non-engaging state and an engaging state. Typically for such applications, support 240 is advanced to right atrium 76 while catheter-engaging element 238 is in the non-engaging state (FIG. 4A), and is subsequently transitioned to the engaging state within the right atrium (FIG. 4B).

Catheter-engaging element 238 may be configured to be transitioned between its open and engaging states by comprising a shape-memory material. For example, the catheter-engaging element may be constrained in the non-engaging state (e.g., by a constraint such as a stiff internal rod or an overtube) and may automatically transition into the engaging state upon removal of the constraint.

Catheter-engaging element 238 may be configured to be transitioned between its non-engaging and engaging states via active bending, such as by pulling one or more pull-wires (such as pull-wires that are used in the art of steerable catheters, mutatis mutandis) that are attached to the catheter-engaging element.

For some applications, catheter-engaging element 238 engages (e.g., contacts) catheter 22 while the catheter-engaging element 238 is in the non-engaging state. For some applications, catheter-engaging element 238 engages catheter 22 while the catheter-engaging element transitions from the non-engaging state to the engaging state.

FIG. 4B shows catheter-engaging element 238 engaging catheter 22 (e.g., supported portion 34 thereof) after distal portion 24 of the catheter has already been advanced through interatrial septum 82 and into left atrium 80. Alternatively, catheter-engaging element 238 may be used to engage catheter 22 while distal portion 24 of the catheter 22 is disposed within right atrium 76 (e.g., before advancing the distal portion through interatrial septum 82). It is hypothesized by the inventors that, in this manner, and for some applications, catheter-engaging element 238 advantageously allows the operator to decide at what stage during the procedure to engage catheter 22.

As described hereinabove in reference to support 40 shown in FIGS. 1C-D, mutatis mutandis, support 240 is used to support catheter 22 (e.g., supported portion 34 thereof) within right atrium 76. Typically, catheter-engaging element 238 supports catheter 22 while the catheter-engaging element is in the engaging state. For some such applications, support 240 is used to apply force to supported portion 34, changing an orientation of the catheter (e.g., an orientation of right atrial portion 46 thereof, FIG. 4C). Typically, support 240 is used to continually apply the force to supported portion 34, such that right atrial portion 46 remains in a tensioned orientation while distal portion 24 is steered to mitral valve 86, as shown in FIGS. 1E-F, mutatis mutandis.

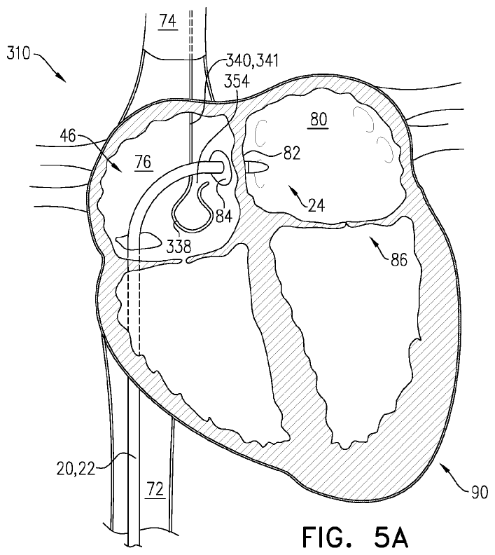

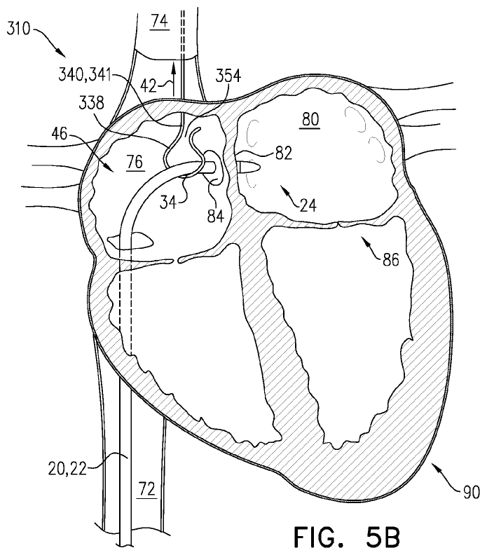

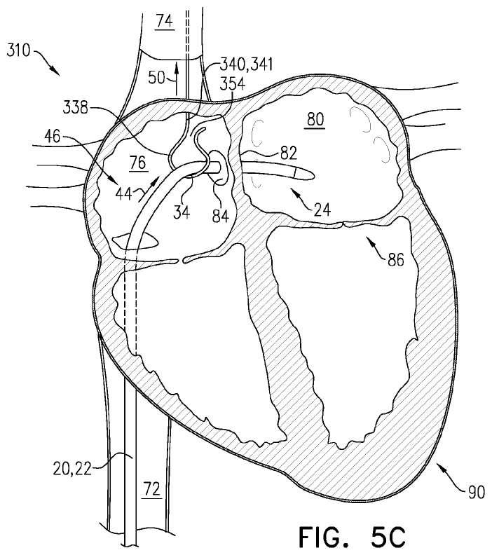

Reference is made to FIGS. 5A-C, which are schematic illustrations showing use of a multi-component system 310 comprising a support 340 and delivery tool 20 to transseptally access left atrium 80 of heart 90 of a subject, in accordance with some applications of the invention.

For some applications, elements of system 310 (e.g., delivery tool 20 and support 340) are packaged together commercially, e.g., in a kit.

Except where noted, system 310 is typically identical to system 10 described hereinabove, and is used similarly as system 10, mutatis mutandis. Components that are identically named between the systems typically share similar features and serve similar functions as each other. For example, system 310 comprises delivery tool 20, similarly to as described hereinabove in reference to FIGS. 1A-1F, mutatis mutandis. As such, the description below of system 310 focuses upon features that are particular to system 310 (e.g., the use of support 340).

FIG. 5A shows a distal portion 341 of support 340, comprising catheter-engaging element 338, having been advanced to right atrium 76. Catheter-engaging element 338 is used similarly to catheter-engaging element 238 described hereinabove, with the exception of catheter-engaging element 338 typically not being transitionable from a non-engaging state to an engaging state. Instead, catheter-engaging element 338 is typically static (e.g., non-transitionable), and is shaped to form a snare. The snare formed by catheter-engaging element 338 typically defines an opening 354 dimensioned to facilitate smooth engaging (e.g., ensnaring) of catheter 22 by the opening being wider than an external diameter of the catheter (e.g., of supported portion 34 thereof). Alternatively, opening 354 may be slightly narrower than the external diameter of the catheter, and may temporarily flex open in response to the catheter being moved through the opening, e.g., to increase stability of the engagement of the catheter. A remainder of catheter-engaging element 338 (a portion not defining opening 354) is typically shaped to facilitate supporting catheter 22 after supported portion 34 passes through the opening and is ensnared.

FIG. 5A shows support 340 disposed within right atrium 76 such that opening 354 is disposed: closer to superior vena cava 74 than the remainder of catheter-engaging element 338, yet further from the superior vena cava than a targeted portion of catheter 22 (e.g., a portion that will become supported portion 34), and adjacent to the targeted portion of the catheter. In this way, moving 42 the catheter-engaging element 338 closer to superior vena cava 74 (FIG. 5B) results in supported portion 34 of catheter 22 having passed through opening 354 of catheter-engaging element 338, supporting (e.g., ensnaring) the catheter. Typically, and as shown, catheter-engaging element 338 is used to ensnare catheter 22 while distal portion 24 of the catheter is disposed in left atrium 80 (e.g., after the distal portion has advanced through interatrial septum 82 and into left atrium 80). Alternatively, catheter-engaging element 338 may be used to ensnare catheter 22 while distal portion 24 of the catheter 22 is disposed within right atrium 76 (e.g., before advancing the distal portion through interatrial septum 82). It is hypothesized by the inventors that, in this manner, and for some applications, catheter-engaging element 338 advantageously allows the operator to decide at which stage during the procedure to engage catheter 22.

For some applications, support 340 is used to apply a force to catheter 22 (e.g., supported portion 34 thereof), thereby supporting the catheter. For some such applications, and as shown, application of the force to supported portion 34, changes an orientation of catheter 22 (e.g., right atrial portion 46 thereof, FIGS. 5B-C).

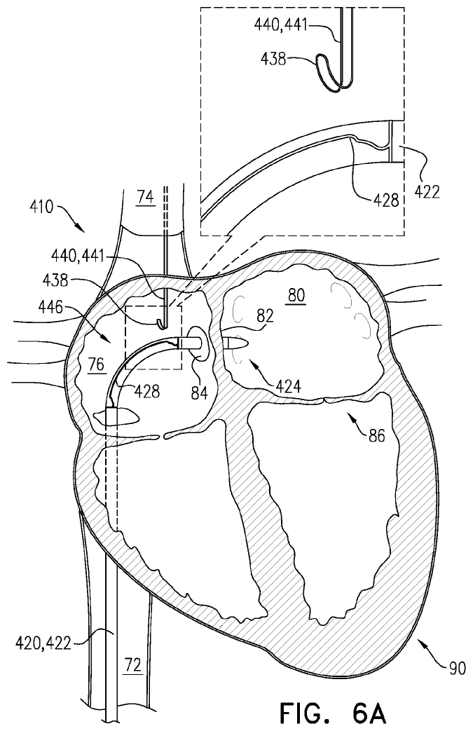

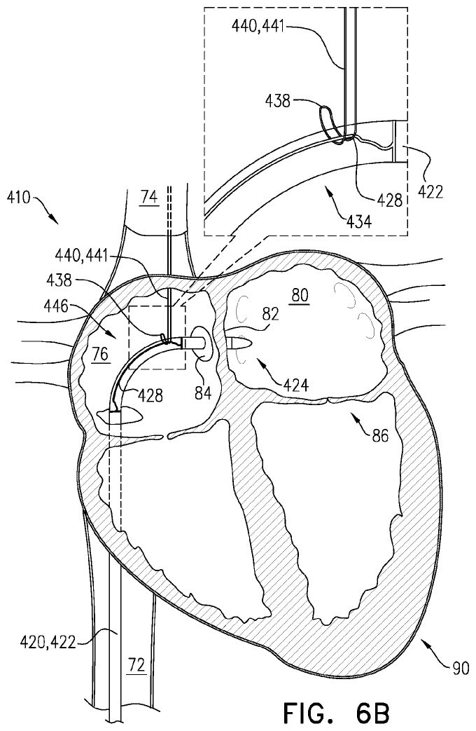

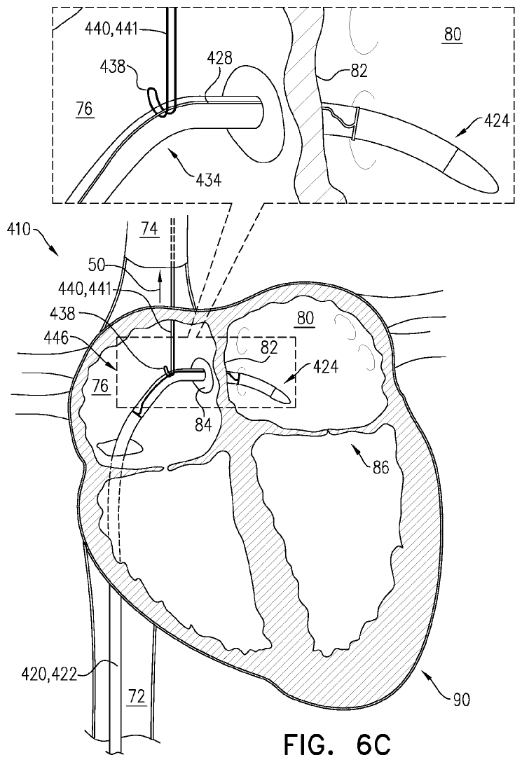

Reference is made to FIGS. 6A-C, which are schematic illustrations showing use of a multi-component system 410 comprising a support 440 and a delivery tool 420 to transseptally access left atrium 80 of heart 90 of a subject, in accordance with some applications of the invention.

For some applications, elements of system 410 (e.g., delivery tool 20 and support 440) are packaged together commercially, e.g., in a kit.

Except where noted, system 410 is typically identical to system 10 described hereinabove, and is used similarly as system 10, mutatis mutandis. Components that are identically named between the systems typically share similar features and serve similar functions as each other. Components bearing identical reference numerals are typically interchangeable between delivery tools 20 and 420. As such, the description below of system 410 focuses upon features that are particular to system 410 (e.g., the use of support 440 and delivery tool 420).

System 410 typically differs from the systems described hereinabove in at least two manners: (i) catheter 422 of delivery tool 420 comprises a support-engaging element 428, and (ii) a distal portion 441 of support 440 comprises a catheter-engaging element 438 dimensioned to engage support-engaging element 428. Support-engaging element 428 is typically a discrete element defined by and/or attached to the main body of the catheter, e.g., coupled to an outer wall of the catheter that is engaged by support 440.

For some applications, and as shown, catheter-engaging element 438 is shaped to define a hook. For some applications, support-engaging element 428 is shaped to define a rail extending along a portion of catheter 422. Typically for such applications, after catheter-engaging element 438 is advanced to right atrium 76 (FIG. 6A), the hook is used to engage (e.g., to ensnare) the rail of support-engaging element 428 (FIG. 6B).

FIG. 6B shows catheter-engaging element 438 engaging support-engaging element 428 while distal portion 424 of catheter 422 is disposed in left atrium 80 (e.g., after the distal portion has advanced through interatrial septum 82 and into left atrium 80). Alternatively, catheter-engaging element 438 may be used to engage support-engaging element 428 while distal portion 424 of catheter 422 is disposed within right atrium 76 (e.g., before advancing the distal portion through interatrial septum 82). It is hypothesized by the inventors that offering the operator the option of using catheter-engaging element 438 to engage support-engaging element 428 at different stages in advancement of distal portion 424 towards mitral valve 86 facilitates responsiveness, on part of the operator, to the subject's particular cardiac anatomy and/or pathophysiology.

For some applications, hooking rail of support-engaging element 428, using the hook formed by catheter-engaging element 438, defines a supported portion 434 of catheter 422 by virtue of the presence of the rail at that portion of the catheter.

For some applications, in contrast to the embodiments described hereinabove, which typically support the catheter at a discrete point defining supported portion 34, a superiorly-directed force applied to the support-engaging element from catheter-engaging element 438 may be typically distributed, by the support-engaging element, along a length of catheter 422 (e.g., along supported portion 434 at which the support-engaging element is disposed).

For some applications, catheter-engaging element 438 is used to move catheter 422 (e.g., supported portion 434 thereof), by applying superiorly-directed force 50 from support 440 to the catheter. For example, the force may change an orientation of catheter 22 (e.g., of right atrial portion 46 thereof), as shown in FIGS. 6C.

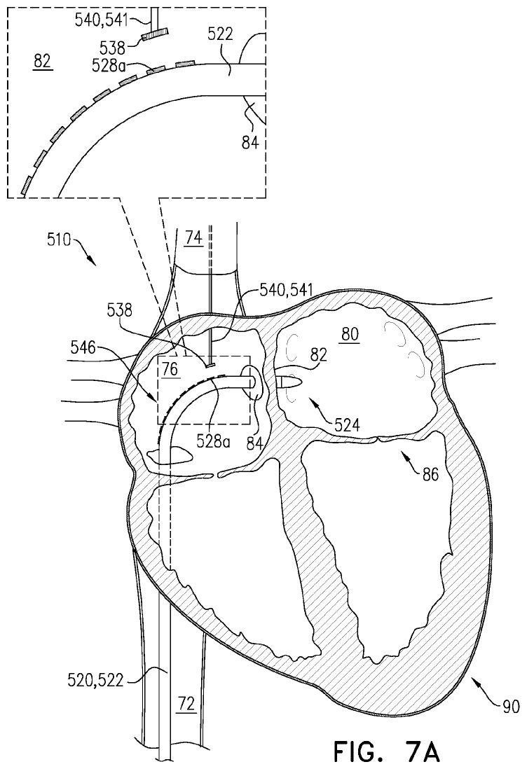

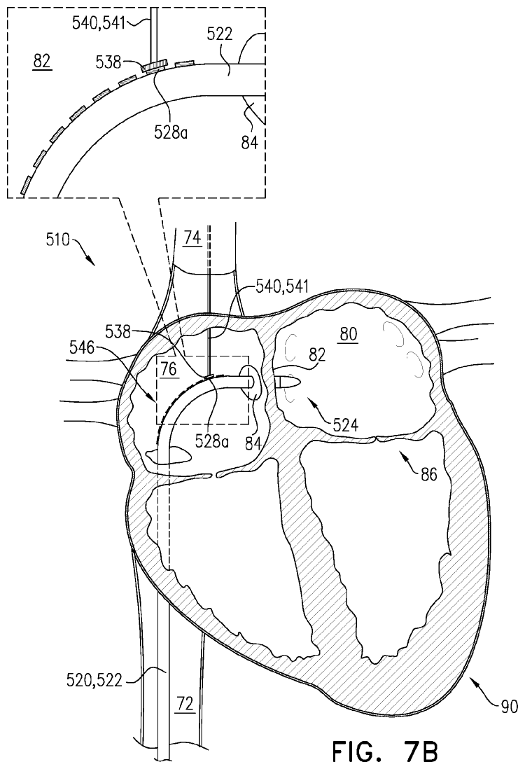

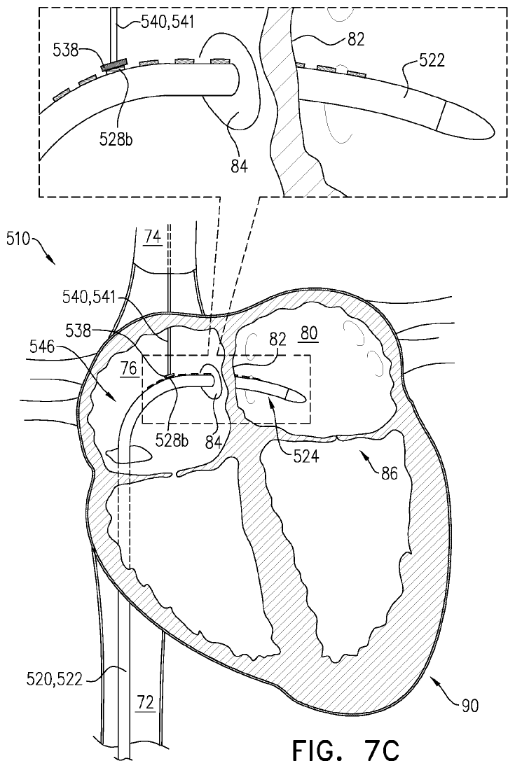

Reference is made to FIGS. 7A-C, which are schematic illustrations showing use of a multi-component system 510 comprising a support 540 and a delivery tool 520 to transseptally access left atrium 80 of heart 90 of a subject, in accordance with some applications of the invention. Except where noted, system 510 is typically identical to system 10 described hereinabove, and is used similarly as system 10, mutatis mutandis.

For some applications, elements of system 510 (e.g., delivery tool 20 and support 540) are packaged together commercially, e.g., in a kit.

In systems described hereinabove, such as system 10, the catheter-engaging element of the support engages catheter 22 mechanically. However, for some applications of the invention, the support engages the catheter by non-mechanical means (e.g., magnetically). For example, in system 510, catheter-engaging element 538 of distal portion 541 of support 540 magnetically engages catheter 522. Besides the difference in the manner by which the catheter-engaging element engages the catheter, components that are identically named between the systems typically share similar features and serve similar functions as each other. As such, the description below of system 510 focuses upon features that are particular to system 510 (e.g., the use of support 540 and delivery tool 520).

As shown in FIG. 7A, and similarly to catheter 422 of system 410 described hereinabove, catheter 522 of system 510 also comprises a support-engaging element 528. However, instead of forming a rail, support-engaging element 528 comprises a magnet. Correspondingly, instead of forming a hook, catheter-engaging element 538 of support 540 also comprises a magnet. For some such applications, catheter-engaging element 538 engages catheter 522 (e.g., support-engaging element 528 thereof) by magnetically engaging the catheter to the catheter-engaging element. For example, catheter-engaging element 538 may comprise an electromagnet. For some such applications, the electromagnet is activated, in order to magnetically engage support-engaging element 528 to catheter-engaging element 538. For example, catheter-engaging element 538 may be an electromagnet that is selectively activated and deactivated by the operator. Alternatively or in addition, support-engaging element 528 may comprise an electromagnet.

For some applications in which catheter-engaging element 538 comprises the electromagnet, support-engaging element 528 comprises a ferromagnetic material. Similarly, for some applications in which support-engaging element 528 comprises the electromagnet, catheter-engaging element 538 comprises a ferromagnetic material.

For some applications, and as shown in FIGS. 7A-C, catheter 522 comprises a plurality of support-engaging elements 528, (including a first support-engaging element labelled as 528a in FIGS. 7A-B, and a second support-engaging element labelled as 528b in FIG. 7C). It is hypothesized by the inventors that the plurality of support-engaging elements 528 facilitates supporting of the catheter using catheter-engaging element 538, by offering the operator a choice of targets (e.g., support-engaging elements) that may be magnetically engaged with the catheter-engaging element.

With the exception of differences described hereinabove between system 410 and system 510, system 510 is used essentially similarly to system 410 described hereinabove. That is, catheter-engaging element 538 engages (e.g., magnetically engages) support-engaging element 528, instead of hooking support-engaging element 428 using catheter-engaging element 438. In this way, a supported portion 534 of catheter 522 is defined by virtue of the supported portion 534 being adjacent to magnet 560 which is magnetically engaged to catheter-engaging element 538.

FIG. 7B shows catheter-engaging element 538 engaging support-engaging element 528a, while distal portion 524 of catheter 522 is disposed in left atrium 80 (e.g., after the distal portion has advanced through interatrial septum 82 and into left atrium 80). Alternatively, catheter-engaging element 538 may be used to engage support-engaging element 528 while distal portion 524 of catheter 522 is disposed within right atrium 76 (e.g., before advancing the distal portion through interatrial septum 82). It is hypothesized by the inventors that offering the operator the option of using catheter-engaging element 538 to engage support-engaging element 528 at different stages in advancement of distal portion 524 towards mitral valve 86 facilitates responsiveness, on part of the operator, to the subject's particular cardiac anatomy and/or pathophysiology.

For some applications, catheter-engaging element 538 is used to serially magnetically engage a plurality of support-engaging elements 528 disposed along an outer wall of catheter 522. Typically for such applications, catheter-engaging element 538 is an electromagnet, selectively activated, deactivated, and reactivated by the operator, in order to: magnetically engage a first support-engaging element 528 (FIG. 7B), cease the magnetic engagement between the catheter-engaging element and the first support-engaging element 528a, and then magnetically engage a second support-engaging element 528b (FIG. 7C). This is typically accomplished while steering distal portion 524 of catheter 522 toward mitral valve 86, thereby supporting catheter 422 in right atrium 76. It is hypothesized by the inventors that serially magnetically engaging support-engaging elements 528 facilitates using support 540 to support catheter 522, by reducing resistance to advancement of the catheter while steering distal portion 524 of catheter 522 toward mitral valve 86.

For some applications, supporting catheter 522 by using catheter-engaging element 538 to serially magnetically engage support-engaging elements 528 may facilitate transfer of superiorly-directed force 50 from support 540 to the catheter. For some such applications, applying the force may change an orientation of catheter 22 (e.g., right atrial portion 46 thereof).

The embodiments described hereinabove of catheter-engaging elements and respective support-engaging elements are not meant to be exclusive, and alternative means of engaging the catheter-engaging element to the support-engaging element (e.g., by fitting a protrusion into a recess) are also contemplated.

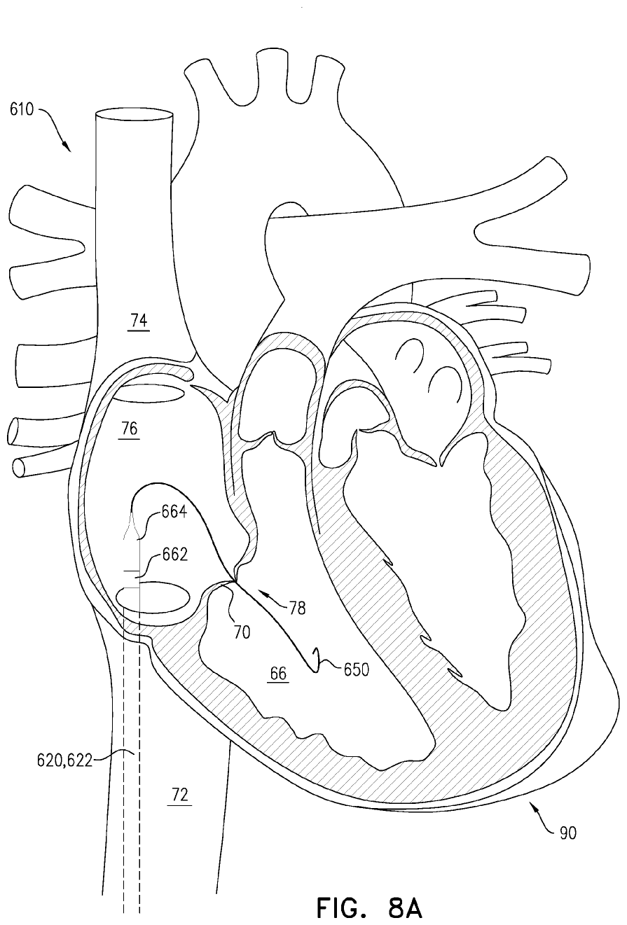

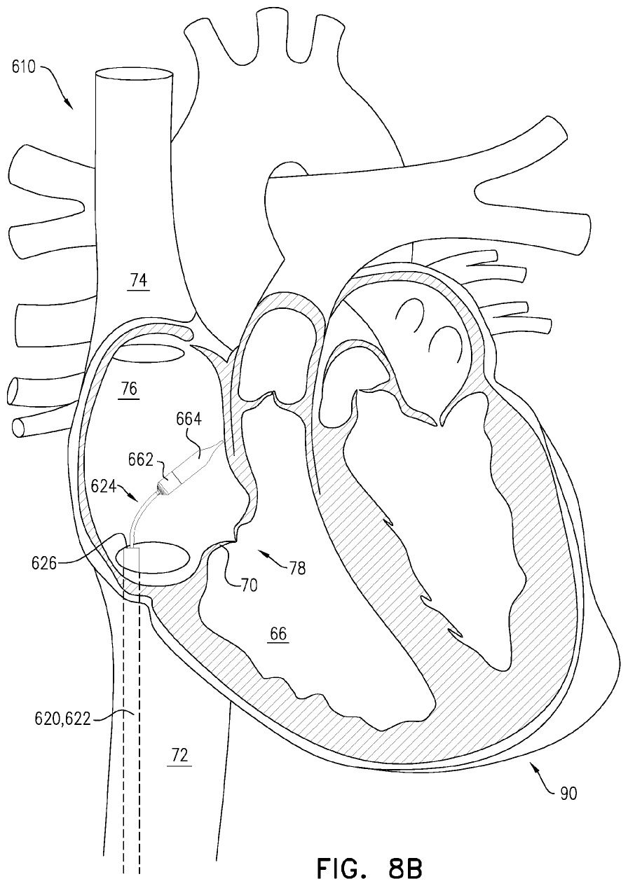

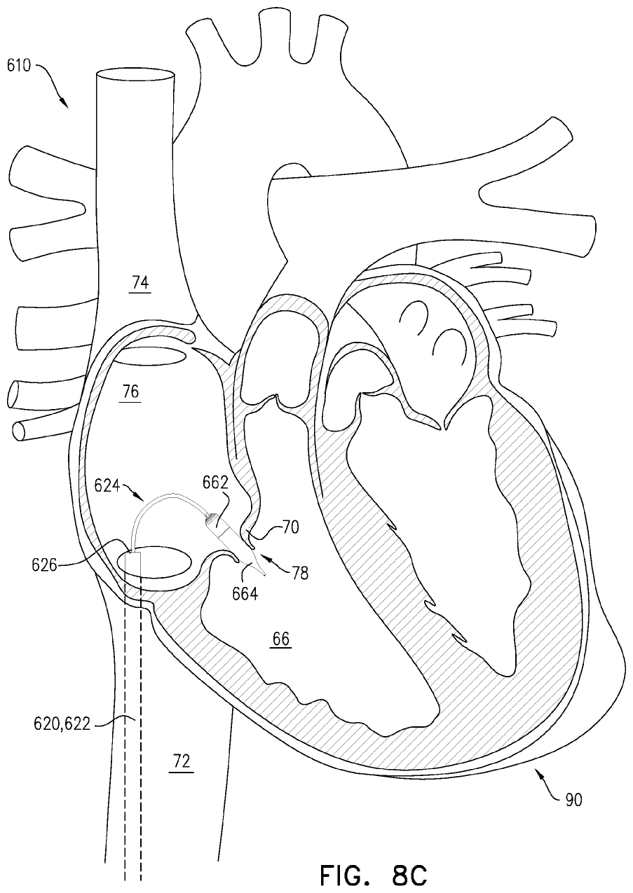

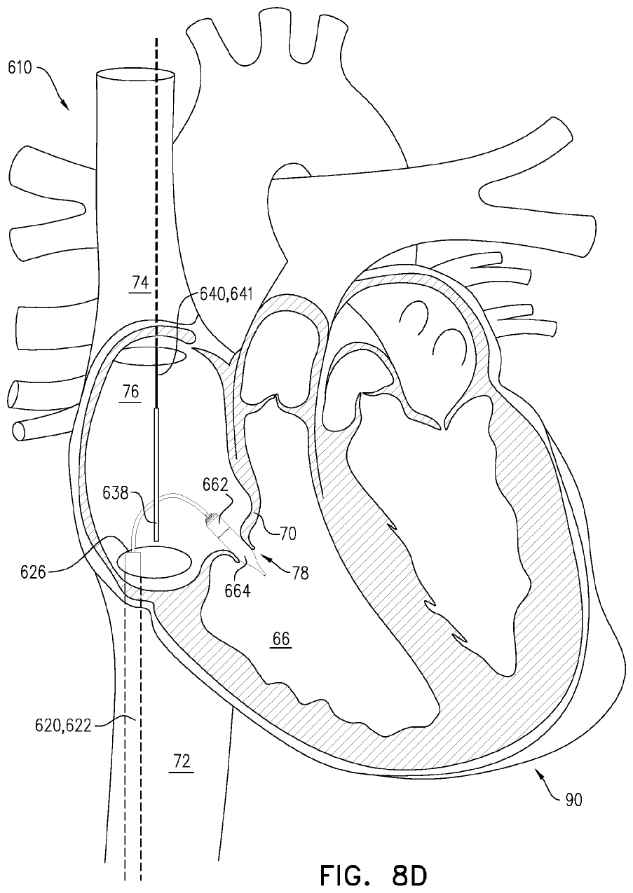

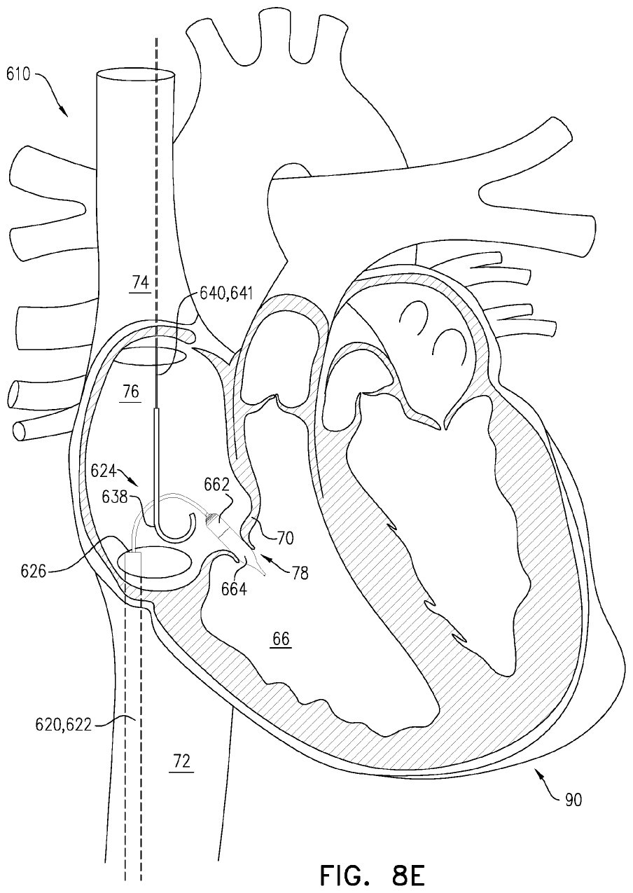

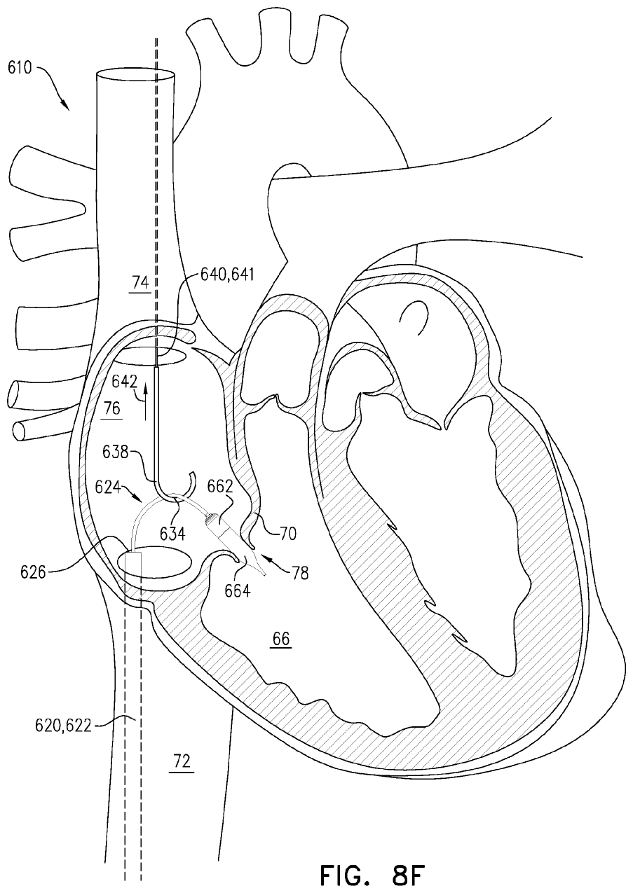

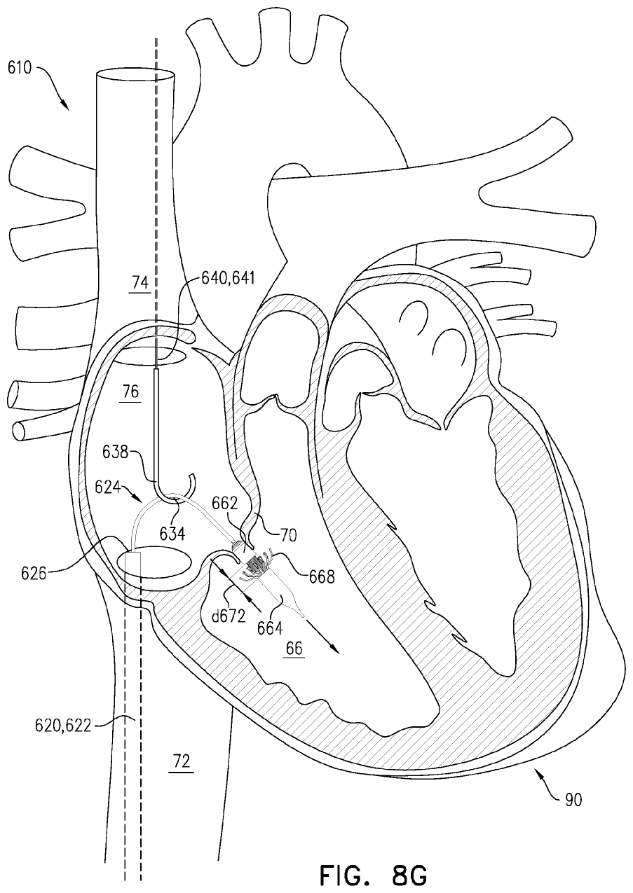

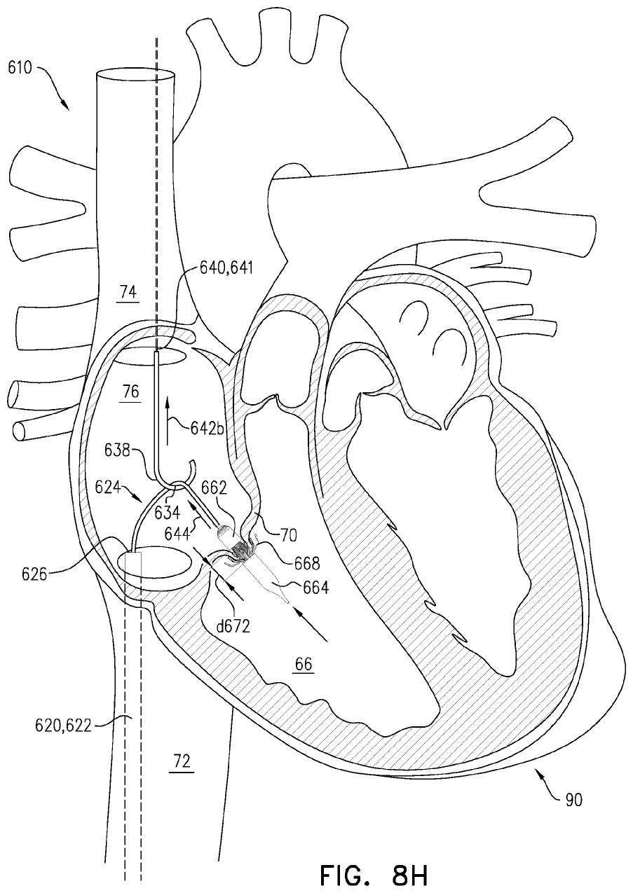

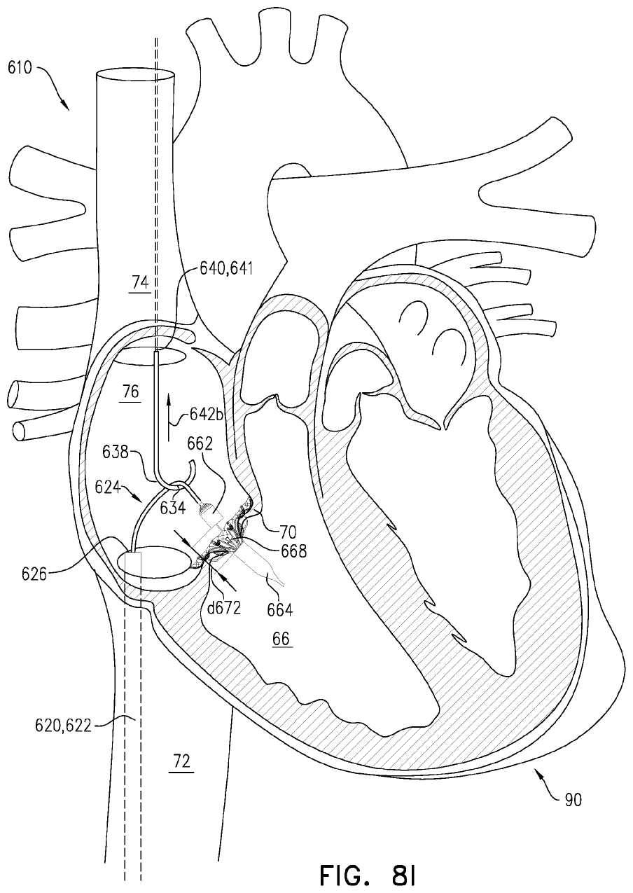

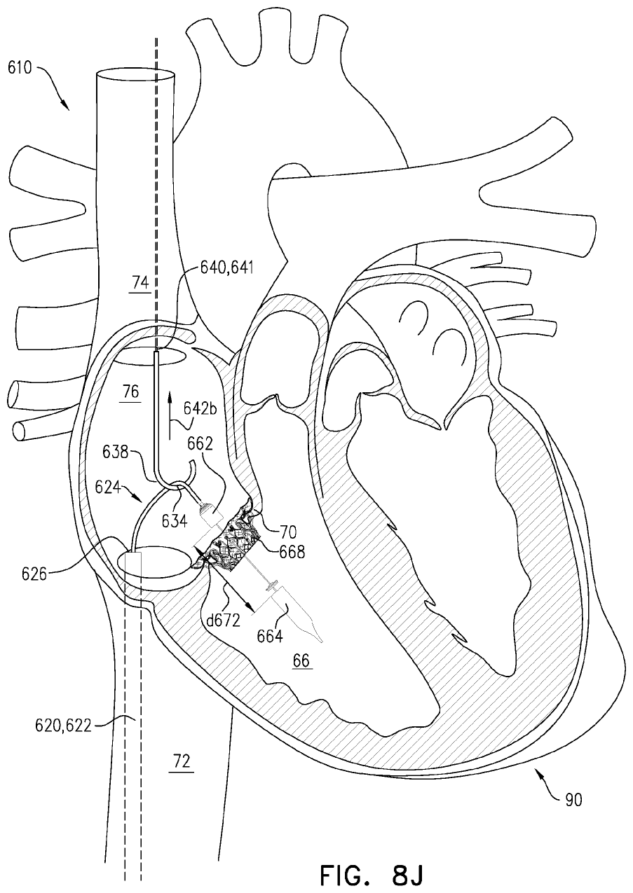

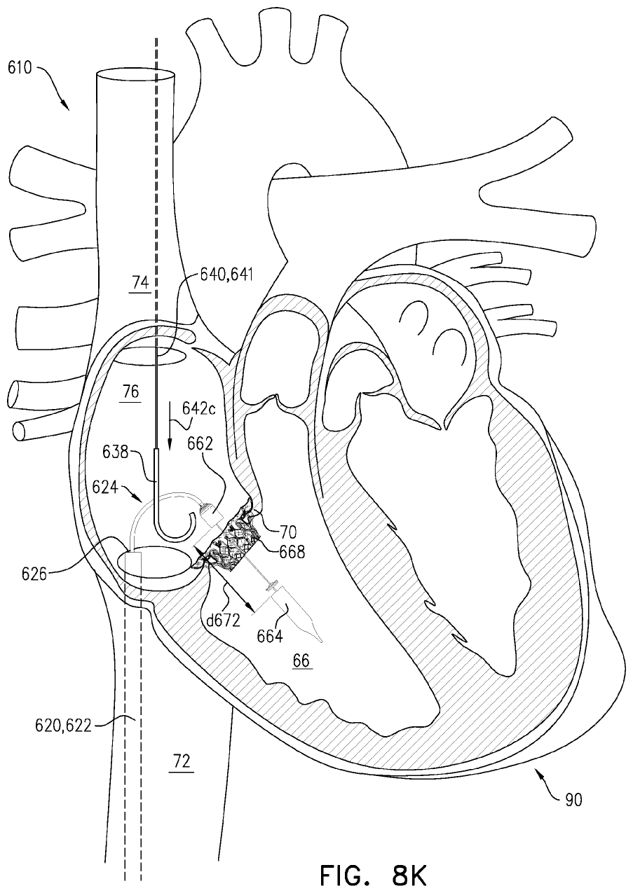

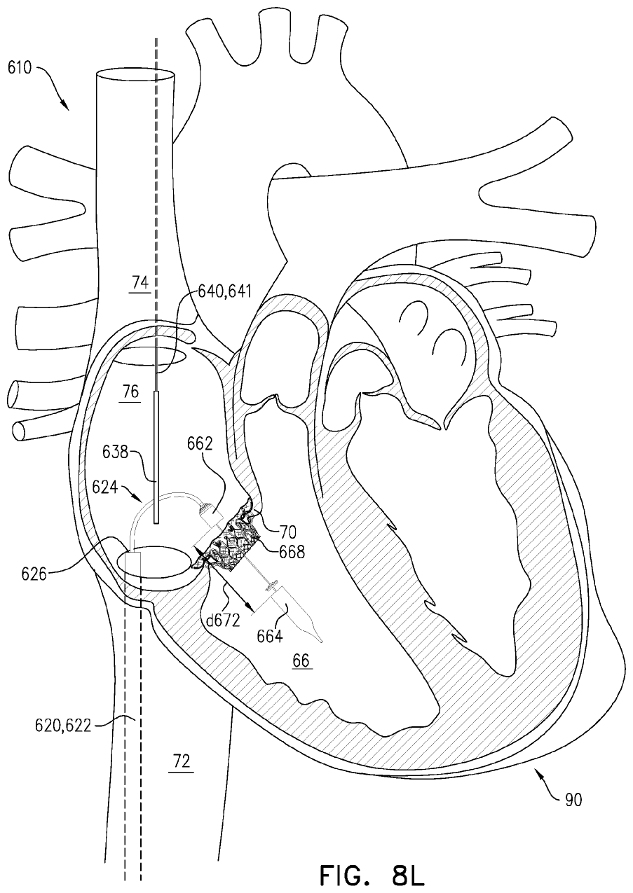

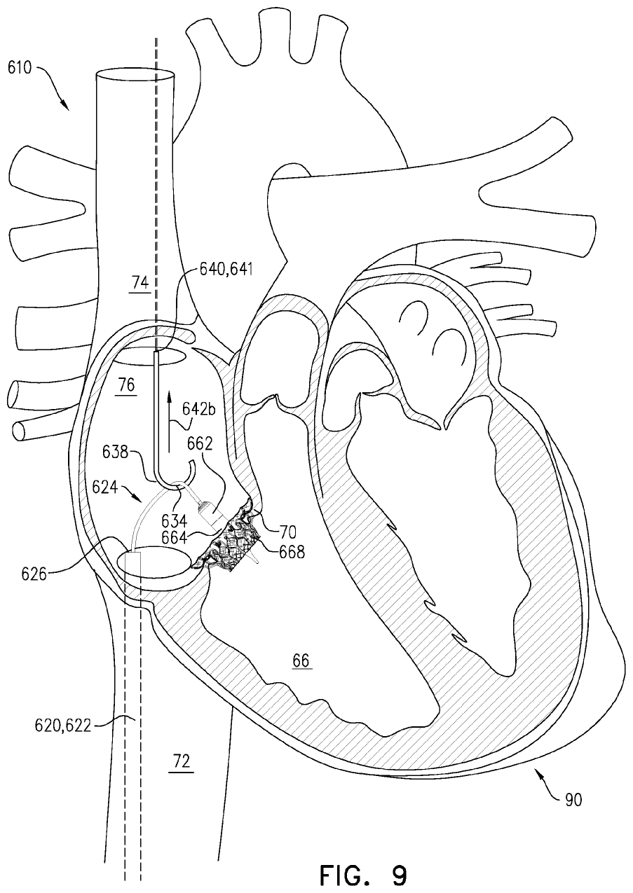

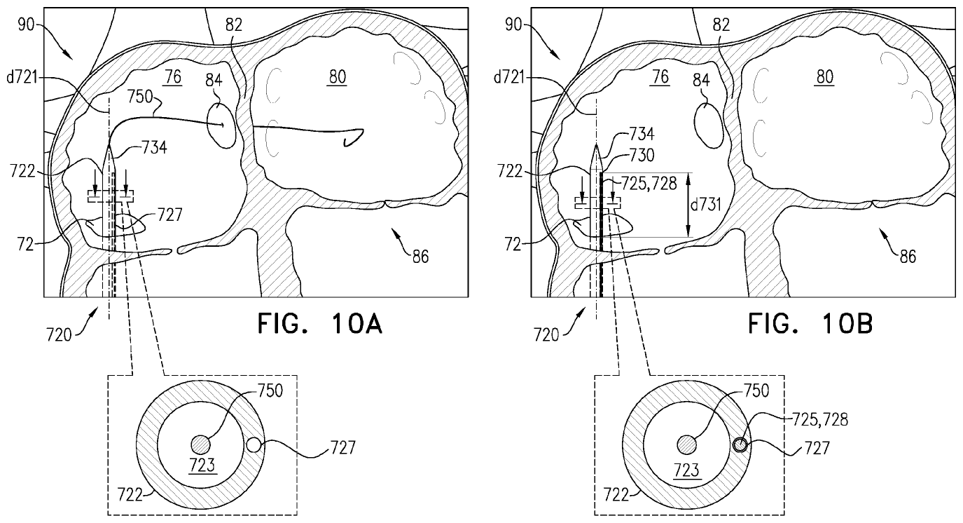

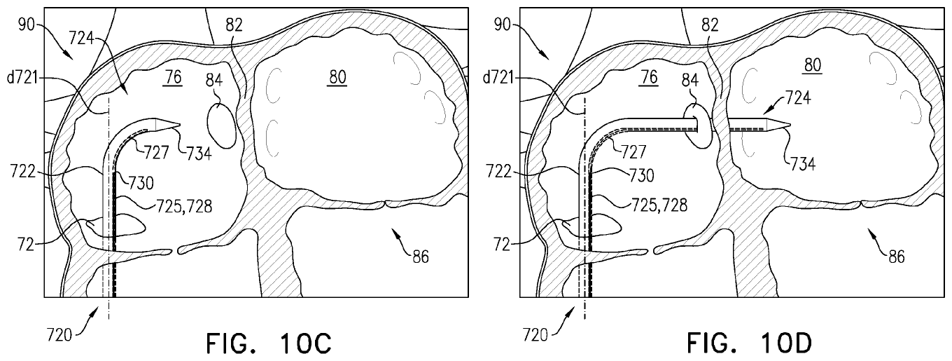

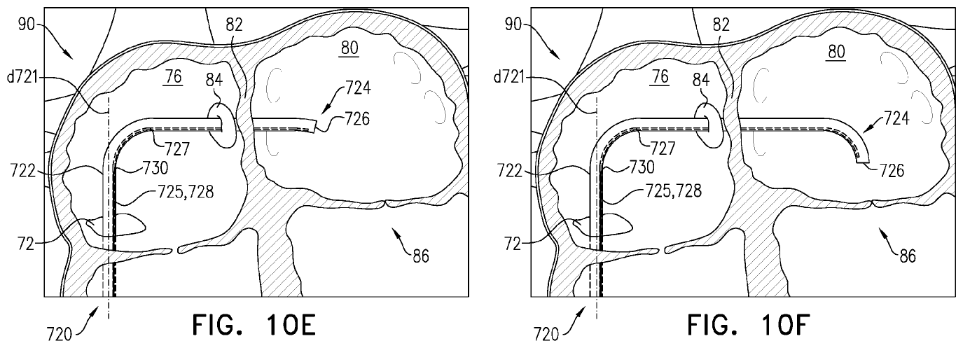

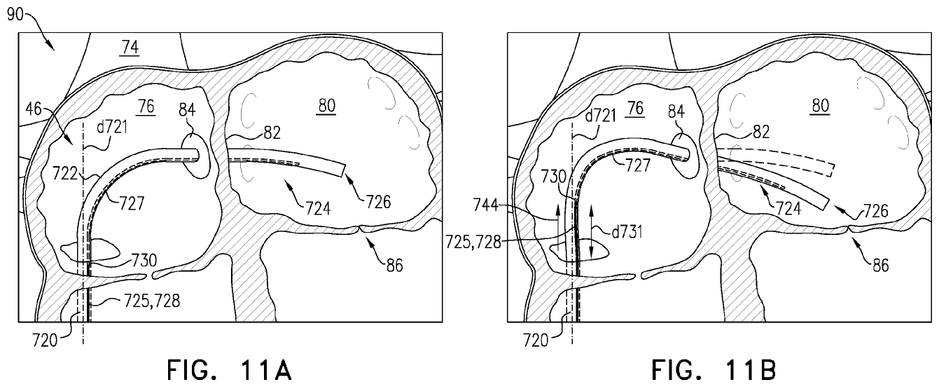

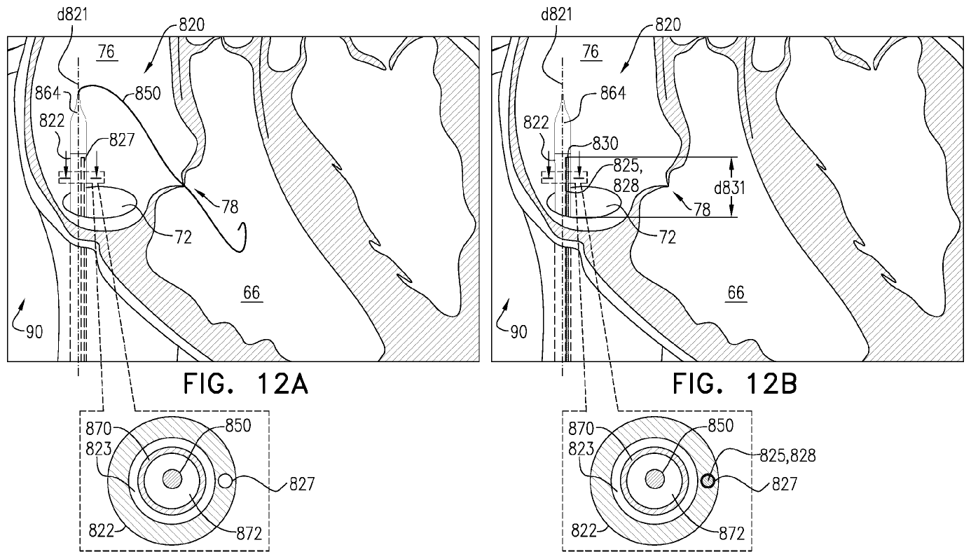

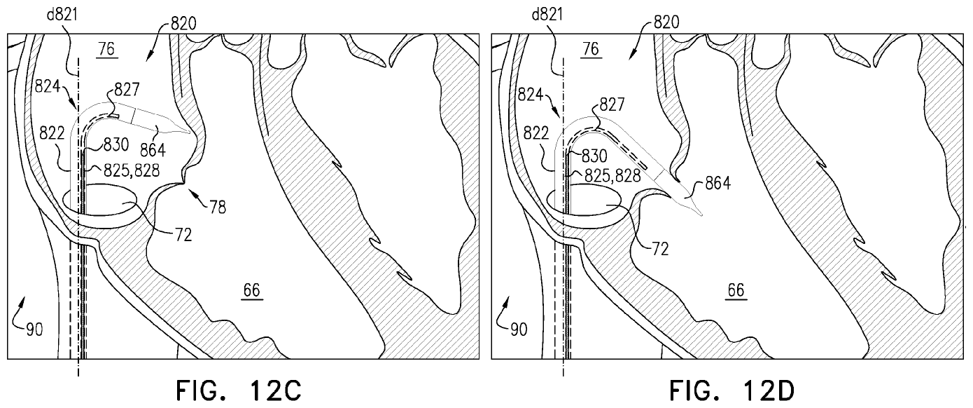

Reference is made to FIGS. 8A-8L, and 9, which are schematic illustrations showing use of a multi-component system 610 comprising a support 640 and delivery tool 620 for transfemorally implanting a prosthetic valve 668 at a native valve of heart 90, in accordance with some applications of the invention. While system 610 is described as being used to implant prosthetic valve 668 at tricuspid valve 78, use of the system to implant the prosthetic valve at other native valves of the heart is also contemplated.

FIG. 8A shows delivery tool 620 having been transfemorally advanced (in accordance with some applications, along a guidewire 650, shown in FIG. 8A but not in FIGS. 8B-8L and 9), via inferior vena cava 72, to right atrium 76 of heart 90. Delivery tool 620 comprises a catheter 622, a proximal housing 662 and a distal housing 664. Typically, delivery tool 620 comprises a steerable distal portion 624, which is shown having been extended and exposed from catheter 622, in FIG. 8B.

It is to be noted that, throughout this application (including the specification and the claims), the terms “steer” and “steerable” refer to active steering of an element such as a catheter, e.g., by using an extracorporeal controller that is operatively and/or mechanically coupled to a steerable portion of the element to effect bending of the steerable portion of the element. (This is in contrast to a flexible but non-steerable element, which may bend in response to encountering forces during advancement through the body of the subject.) Steerability of distal portion 624 facilitates steering of distal housing 664 and proximal housing 662 toward tricuspid valve 78 (FIGS. 8B-C). Typically, and as shown in FIG. 8C, distal portion 624 is further advanced, such that at least a portion of distal housing 664 is disposed within right ventricle 66.

FIG. 8D shows a distal portion 641 of support 640 having been advanced via superior vena cava 74 to right atrium 76. Support 640 as shown is in many ways similar to support 240 described herein below in reference to FIGS. 4A-C. For example, support 640 comprises a catheter-engaging element 638, which has a non-engaging state and an engaging state. However, the scope of the invention includes using other support described herein, mutatis mutandis.

Typically, support 640 is used to engage delivery tool 620 within right atrium 76. For some applications, and similarly to support 240 described hereinabove, support 640 is advanced to right atrium 76 while catheter-engaging element 638 is in a non-engaging state (FIG. 8D), after which catheter-engaging element 638 transitions into the engaging state (FIG. 8E). For some applications, support 640 engages delivery tool 620 by transitioning catheter-engaging element 638 from the non-engaging state to the engaging state (FIG. 8F). Alternatively or in addition, a force (e.g., pulling force 642) may be applied to support 640, such that catheter-engaging element 638 engages delivery tool 620 (e.g., distal portion 624 thereof).

For some applications, support 640 is used to support distal portion 624 in right atrium 76 while delivery tool 620 is steered toward tricuspid valve 78. Typically for such applications, catheter-engaging element 638 is used to support catheter 22 while in the engaging state. It is hypothesized by the inventors that this facilitates steering distal portion 624 toward tricuspid valve 78, as described hereinabove in reference to FIGS. 2B-C, mutatis mutandis, such that supported portion 634 may serve as a pivot point with which one or more forces (e.g., a superiorly-directed force applied by support 640 and a pushing force applied to delivery tool 620) can interact.

Typically, support 640 is used to support delivery tool 620 by applying a force (e.g., a superiorly-directed force) to the delivery tool (e.g., to distal portion 624 thereof). For some applications, applying the force causes support 640 (e.g., catheter-engaging element 638 thereof) to engage delivery tool (e.g., distal portion 624 thereof), defining an engaged portion 634.

Subsequently, prosthetic valve 668 is deployed from delivery tool 620 at the native valve (e.g., tricuspid valve 78). For some applications, and as shown in FIG. 8G, prosthetic valve 668 is deployed by increasing a distance d672 between proximal housing 662 and distal housing 664. For example, distal housing 664 may be moved distally with respect to proximal housing 662, and/or the proximal housing may be moved proximally with respect to the distal housing. In this way, movement of proximal housing 662 and distal housing 664 facilitates exposure of prosthetic valve 668 from the housings.

For some applications, prosthetic valve 668 is comprised of a shape-memory superelastic material (e.g., Nitinol), such that exposure of the prosthetic valve from the housings causes the prosthetic valve to automatically expand. Alternatively, prosthetic valve 668 may require an expansion element (e.g., an inflatable balloon, not shown) in order to expand following deployment from the housings, mutatis mutandis.

FIG. 8H shows application of a superiorly-directed force 642b to supported portion 634 of distal portion 624, using support 640. For some applications, the force is applied while prosthetic valve 668 is at least partially disposed within right ventricle 66 of heart 90. For example, and as shown, the force is applied while prosthetic valve 668 is partially deployed from delivery tool 620.

For some applications, applying the superiorly-directed force causes distal portion 624 to move. For some applications and as shown, application of the superiorly-directed force moves prosthetic valve 668, proximal housing 662 and distal housing 664 in an upstream direction (indicated by arrow 644). For example, prosthetic valve 668 is moved proximally while the prosthetic valve is partially deployed. For example, movement of prosthetic valve 668 in an upstream direction while ventricular snares of the prosthetic valve are in an expanded state facilitates coupling the prosthetic valve to tissue 70 (e.g., leaflets) of the tricuspid valve (FIG. 8H). It is hypothesized by the inventors that such upstream movement of prosthetic valve 668 is facilitated by support 640, e.g., compared to using delivery tool 620 alone. It is further hypothesized by the inventors that such facilitation is particularly useful in instances in which the orientation of the prosthetic valve during its upstream movement, and the vector of the upstream movement, are important for successful implantation.

FIG. 8I shows prosthetic valve 668 being further deployed while superiorly-directed force 642b continues to be applied to distal portion 624 using support 640. As shown in FIG. 8J, prosthetic valve 668 is fully deployed at tricuspid valve 78. It is hypothesized by the inventors that applying superiorly-directed force 642b to delivery tool 620 using support 640, while deployment of prosthetic valve 668 is completed, facilitates coupling of the prosthetic valve to ventricular tissue 70.

Typically, and as shown in FIG. 8K, following deployment (e.g., subsequently to full deployment) of prosthetic valve 668, a disengaging force (e.g., pushing force 642c) is applied to support 640. In this way, support 640 disengages delivery tool 620, such that force is no longer transferred from support 640 to delivery tool 620. For some such applications, support 640 is disengaged from delivery tool 620 by transitioning catheter-engaging element 638 from the engaging state to the non-engaging state (FIG. 8L). Subsequently to disengaging support 640 delivery tool 620, the support is typically withdrawn (e.g., via superior vena cava 74). Further typically, subsequently to disengaging support 640 from delivery tool 620, distal housing 664 is retracted proximally through the prosthetic valve, facilitating withdrawal of delivery tool 620 proximally via inferior vena cava 72.

For some applications, after implantation of prosthetic valve 668, support 640 is used to facilitate withdrawal of a portion of delivery tool 620 (e.g., of housing 664) into the right atrium, by again applying the superiorly-directed force, prior to disengagement of the support from the delivery tool, e.g., as shown in FIG. 9.

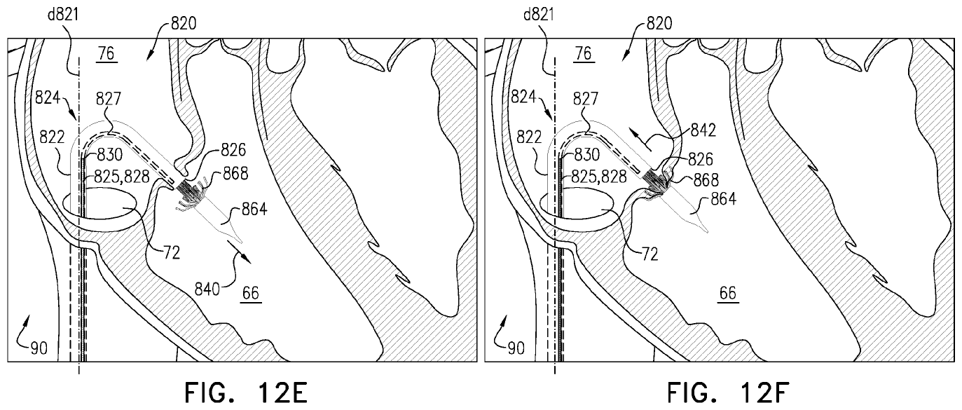

Reference is made to FIGS. 10A-F and 11A-B, which are schematic illustrations showing use of a delivery tool 720 comprising a catheter 722 and a stylet 725 for transseptally accessing left atrium 80, in accordance with some applications of the invention.

For some applications, elements of delivery tool 720 (e.g., catheter 722 and stylet 725) are packaged together commercially, e.g., in a kit.

Except where noted, delivery tool 720 is used similarly as delivery tool 20, mutatis mutandis. Components that are identically named between the systems typically share similar features and serve similar functions as each other. For example, delivery tool 720 comprises catheter 722, similarly to catheter 22 described hereinabove in reference to FIGS. 1A-1F, mutatis mutandis. One notable difference differentiating delivery tool 720 from delivery tool 20 is the presence and use of stylet 725, which may at least partially obviate use of support 40. As such, the description below of delivery tool 720 focuses upon features that are particular to delivery tool 720 (e.g., the use of stylet 725).

The presence of the stylet within a portion of catheter 722 increases rigidity of that portion of the catheter. That is, stylet 725 increases the ability of catheter 722 (or portions thereof) to withstand bending forces. For some applications, upon extending stylet 725 within catheter 722, a stiffness of the catheter becomes at least ten percent greater than the stiffness of the catheter alone.

Typically, stylet 725 is itself stiffer than catheter 722, e.g., having a bending stiffness, measured in N/m, that is at least 10% greater than the stiffness of the catheter. Alternatively, stylet 725 may contribute to increased rigidity of catheter 722 without being, itself, stiffer than the catheter. Stylet 725 may be a monolithic rod, e.g., comprising a metal or rigid polymer.

For some applications, delivery tool 720 is used to deliver an implant to left atrium 80 (e.g., to mitral valve 86). Typically for such applications, the implant is advanced within catheter 722, via inferior vena cava 72, to right atrium 76. For example, the implant may be disposed within catheter 722, e.g., until the implant is deployed at mitral valve. For some such applications, the implant may be a prosthetic heart valve or a valve repair device, such as an annuloplasty device.

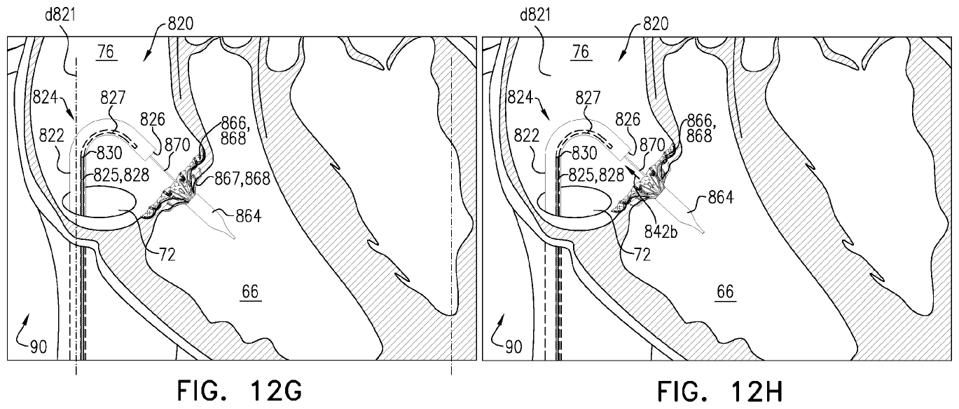

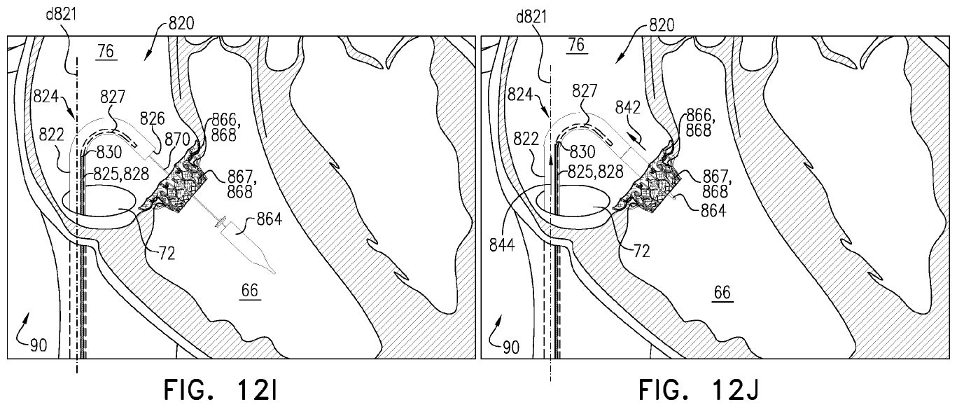

As shown, delivery tool 720 is typically positioned (e.g., advanced transluminally along a guidewire 750, shown in FIG. 12A but not in FIGS. 12B-12J) such that catheter 722 extends, via inferior vena cava 72, to right atrium 76 of heart 90 (FIG. 10A). Further typically, catheter 722 extends along a catheter-advancement axis d721. Catheter 722 typically defines a secondary lumen 727, within which stylet 725 is slidable. For some applications, and as shown, lumen 727 extends from a proximal region of catheter 722 but ends before the distal end of the catheter. As shown in the inset of FIG. 10A, secondary lumen 727 runs parallel to primary lumen 723 that is defined by a wall of catheter 722. For some applications, and as shown, guidewire 750 passes through primary lumen 723. Alternatively, guidewire 850 may pass through a tertiary lumen (not shown) that is defined by a wall of catheter 722, mutatis mutandis.

As shown, catheter 722 is advanced into right atrium 76, to an atrial height d731 in right atrium 76. Height d731 thereby represents the distance by which stylet 725 is extended into right atrium 76. For some applications, atrial height d731 may be between 1 and 70 mm (e.g., between 1 and 10 mm, or between 10 and 70 mm).

As shown in FIG. 10A, catheter 722 may be advanced to right atrium 76 while catheter 722 (e.g., secondary lumen 727 thereof) is empty of stylet 725, after which the stylet (e.g., supporting portion 728 thereof) is extended within the secondary lumen (FIG. 10B). In this way, catheter 722 has a dynamically variable longitudinal stiffness (i.e., the stiffness of the catheter is changeable while the catheter is within heart 90 by adjusting the location of stylet 725 within catheter 722). It is hypothesized by the inventors that: (i) catheter 722 having a lesser stiffness while the catheter is advanced to the heart facilitates navigation through the vasculature, and (ii) at least a portion of the catheter having a greater stiffness facilitates (e.g., supports) use of the catheter to access left atrium 80, as described hereinbelow. Alternatively, catheter 722 may be advanced to right atrium 76 while stylet 725 is already disposed within the catheter (e.g., within secondary lumen 727).

As shown in FIGS. 10C-D, a steerable distal portion 724 of catheter 722 is steered away from axis d721, and through interatrial septum 82. Typically, and as shown, distal portion 724 is steered while distal end 730 of stylet 725 remains in right atrium 76, within catheter 722. That is, advancement of catheter 722 toward and through septum 82 progressively slides the catheter distally off of stylet 725. For some such applications, distal end 730 of stylet 725 remains at a generally constant atrial height d731 while distal portion 724 is steered through interatrial septum 82. Stylet 725, and portions of catheter 722 within which the stylet remains disposed, resist being bent away from axis 721 in response to steering of distal portion 724 of the catheter.

FIG. 10D shows a distal end of catheter 722 having advanced through interatrial septum 82 (e.g., fossa ovalis 84 thereof) and into left atrium 80. For some applications, a piercing device and/or a dilator 734 extends distally out of the distal end of catheter 722, e.g., to facilitate piercing and/or dilating septum 82. For example, the piercing device may comprise a needle, and the needle is used to mechanically puncture the septum. Other methods of piercing septum 82 (e.g., radiofrequency ablation and/or ultrasound ablation) are also contemplated.

FIGS. 10E-10F show distal portion 724 of catheter 722, disposed within left atrium 80, being steered toward mitral valve 86. Typically, and as shown, the catheter is steered while distal end 730 of stylet 725 remains in right atrium 76. For some such applications, distal end 730 of stylet 725 remains at a generally constant atrial height d731 while distal portion 724 is steered toward mitral valve 86.

For some applications, and as shown in FIG. 11A, catheter 722 (e.g., steerable distal portion 724 thereof) is advanced through interatrial septum 82 prior to advancing stylet 725 into right atrium 76. For some such applications, and as shown in FIG. 11B, stylet 725 (e.g., supporting portion 728 thereof) is extended within catheter 722 (e.g., through secondary lumen 727) to atrial height d731, while distal portion 724 is within left atrium 80. For example, supporting portion 728 may be extended into right atrium 76 by pushing (arrow 744) stylet 725 (e.g., by pushing extracorporeally upon a proximal portion of the stylet).

For some applications, and as shown, extending stylet 725 within catheter 722 and into right atrium 76 applies a supporting force to distal portion 724 of the catheter, thereby changing an orientation of the distal portion. In this way, distal opening 726 of the catheter advantageously tilts towards mitral valve 86. For some such applications, extending stylet 725 within catheter 722 and into right atrium 76 changes the orientation of distal portion 724 of the catheter, such that such that distal opening 726 advantageously faces mitral valve 86. It is hypothesized by the inventors that, for some applications, such tilting further facilitates access to the mitral valve, in addition to steering of distal portion 724.

It is hypothesized by the inventors that the use of stylet 725 as described, provides similar advantages to those described hereinabove as being provided by the use of a support, mutatis mutandis. In fact, for some applications, stylet 725 may be considered to be an intracatheter support, while the supports described with reference to FIGS. 1A-9, may be considered to be extracatheter supports.

As described hereinabove in reference to delivery tool 20, accessing left atrium 80 of heart 90 using delivery tool 720 may facilitate performance of one or more clinical interventions, including but not limited to mitral annuloplasty, mitral chord repair, mitral valve replacement, left atrial appendage occlusion, or ablation for atrial fibrillation. Typically for such applications, stylet 725 (e.g., supporting portion 728 thereof) remains in right atrium 76 (e.g., within secondary lumen 727), while the clinical intervention is performed. It is hypothesized by the inventors that tilting distal opening 726 of catheter 722 towards mitral valve 86, e.g., such that the distal opening faces the mitral valve, facilitates performance of the clinical interventions.

Reference is made to FIGS. 12A-J, which are schematic illustrations showing use of a delivery tool 820 comprising a catheter 822 and a stylet 825 for transfemorally implanting a prosthetic valve 868 at native valve of the heart, in accordance with some applications of the present invention. Although delivery tool 820 is described as being used to implant prosthetic valve 868 at tricuspid valve 78, use of the system to implant the prosthetic valve at other native valves of the heart is also contemplated.

Delivery tool 820 is similar in certain aspects to delivery tool 720. Components that are identically named between the systems typically share similar features and serve similar functions as each other. For example, delivery tool 820 comprises catheter 822 and stylet 825, similarly to catheter 722 and stylet 725, mutatis mutandis. As such, the description below of delivery tool 820 focuses upon features that are particular to delivery tool 820.

For some applications, elements of delivery tool 820 (e.g., catheter 822 and stylet 825) are packaged together commercially, e.g., in a kit.

FIG. 12A shows delivery tool 820 having been transfemorally advanced along a guidewire 850, via inferior vena cava 72, to right atrium 76 of heart 90. Similarly to as described hereinabove in reference to catheter 722 of delivery tool 720, catheter 822 of delivery tool 820 typically defines a secondary lumen 827, within which stylet 825 is slidable. Further similarly, and as shown, lumen 827 extends from a proximal region of catheter 822 but ends before the distal end of the catheter.

As shown in the inset of FIG. 12A, secondary lumen 827 is typically thinner than a primary lumen 823 that is defined by catheter 822. Also shown in the inset of FIG. 12A is a shaft 870 that passes through primary lumen 823 of catheter 822. For some applications, shaft 870 defines a shaft lumen 872 that extends from a proximal portion of delivery tool 820, through catheter 822, to prosthetic valve 868. For some such applications, and as shown, guidewire 850 passes through shaft lumen 872, from the proximal portion of delivery tool 820 to a distal end of catheter 822. Alternatively, guidewire 850 may pass through a tertiary lumen (not shown) that is defined by a wall of catheter 822, mutatis mutandis.

FIG. 12B shows supporting portion 828 of stylet 825 extended within secondary lumen 827 of catheter 822, to right atrium 76, e.g., as far as an atrial height d831. Height d831 thereby represents the distance by which stylet 725 is extended into right atrium 76. For some applications, atrial height d831 may be between 1 and 70 mm (e.g., between 1 and 10 mm or between 10 and 70 mm.

Typically, catheter 822 comprises a steerable distal portion 824, which is shown in FIG. 12C as having been extended from stylet 825 and towards tricuspid valve 78. In FIG. 12D, distal portion 824 is shown as having been further advanced, such that at least a portion of a distal housing 864 is steered through tricuspid valve 78, to within right ventricle 66. As described in reference to delivery tool 720, steering of catheter 822 toward and through tricuspid valve 78 progressively slides catheter 822 distally off of stylet 825. For some such applications, distal end 830 of stylet 825 remains at a generally constant atrial height d831 while distal portion 824 is steered through tricuspid valve 78.

Stylet 825 is typically stiffer than catheter 822, as discussed hereinabove in reference to stylet 725, such that the presence of the stylet within catheter 822 increases rigidity of the portion of the catheter in which the stylet is disposed. Therefore, stylet 825, and portions of catheter 822 within which the stylet remains disposed, resist being bent away from axis d821 in response to steering of distal portion 824 of the catheter.

For some applications, and as shown, distal housing 864 is used to house an implant (e.g., prosthetic valve 868) until deployment at the native valve (e.g., tricuspid valve 78). For some applications, and as shown, deployment of prosthetic valve 868 from distal housing 864 is facilitated by distal advancement (840, FIG. 12E) of the distal housing, relative to a distal opening 826 of catheter 822. For example, and as shown, prosthetic valve 868 may comprise a shape-memory material which automatically expands upon exposure from within distal housing 864.

Typically and as shown, prosthetic valve 868 is deployed from delivery tool 820 at the native valve (e.g., tricuspid valve 78) while stylet 825 remains within catheter 822, such that supporting portion 828 of the stylet is within right atrium 76. For example and as shown, distal end 830 of stylet 825 may generally remain disposed at atrial height d831 while prosthetic valve 868 is deployed.

FIG. 12F shows application of a proximally-directed force 842 to catheter 822. Typically for such applications, proximally-directed force 842 is applied while stylet 825 (e.g., supporting portion 828 and distal end 830 thereof) remains disposed, within catheter 822, within right atrium 76, such that the catheter (e.g., portion 824 thereof) slides proximally over the stylet. For some applications, stylet 825 remains stationary with respect to the anatomy while proximally-directed force 842 is applied. Further typically and as shown, proximally-directed force 842 is applied while prosthetic valve 868 is partially deployed from distal housing 864. Similarly to as described hereinabove in reference to application of superiorly-directed force 642b to delivery tool 620, proximally-directed force 842 causes prosthetic valve 868 and distal housing 864 to move in an upstream direction while prosthetic valve 868 is partially deployed.

For some applications, while proximally-directed force 842 is applied, a pull-wire (e.g., that passes through shaft 870, from the proximal portion of delivery tool 820 to prosthetic valve 868) is pulled, in order to adjust an angle at which the prosthetic valve approaches tissue of the native valve. For example, movement of prosthetic valve 868 in an upstream direction, and at a desirable angle, while the ventricular snares of prosthetic valve 868 are in an expanded state facilitates coupling the prosthetic valve to tissue (e.g., leaflets) of tricuspid valve 78 (FIG. 12F).

It is hypothesized by the inventors that the upstream movement of prosthetic valve 868 is facilitated by an interaction between proximally-directed force 842 and rigidity that stylet 825 (e.g., supporting portion 828 thereof) provides to catheter 822. It is further hypothesized by the inventors that such facilitation is particularly useful in instances in which the orientation of the prosthetic valve during its upstream movement, and the vector of the upstream movement, are important for successful implantation.

FIG. 12G shows prosthetic valve 868 being further deployed from distal housing 864, such that shaft 870 is exposed. Typically, shaft 870 is reversibly coupled (e.g., at a distal portion of the shaft), to prosthetic valve 868 and to distal housing 864.

As prosthetic valve 868 is further deployed from distal housing 864, upstream skirt 866 is exposed from distal opening 826 and expands on an atrial side of tricuspid valve 78. In some cases, and as shown in FIG. 12G, expansion of upstream skirt 866 on the atrial side exerts a distal force upon prosthetic valve 868, such that the prosthetic valve becomes disposed undesirably far within right ventricle 66. In some such cases, the prosthetic valve being disposed undesirably far within right ventricle 66 causes straining or stretching of tissue of the native valve.

Typically for such cases, a pulling force 842b (FIG. 12H) is then applied to shaft 870 (e.g., to a proximal portion thereof), which pulls prosthetic valve 868 towards right atrium 76, thereby alleviating the straining or stretching of the tissue.

For some applications, pulling force 842b continues to be applied to shaft 870 while prosthetic valve 868 is fully deployed at tricuspid valve 78 (FIG. 121). For some such applications, shaft 870 detaches from distal housing 864 and/or prosthetic valve 868 in order to facilitate full deployment of the prosthetic valve. It is hypothesized by the inventors that applying pulling force 842b to prosthetic valve 868 while deployment of the prosthetic valve is completed further facilitates coupling of the prosthetic valve to tricuspid valve 78.

Typically, following deployment of prosthetic valve 868 at the native valve, delivery tool 820 is withdrawn by retracting distal housing 864 through the prosthetic valve (FIG. 12J). For some such applications, and as shown, a pushing force 844 is applied (e.g., extracorporeally applied) to stylet 825, e.g., while proximally-directed force 842 is applied to catheter 822. As shown, pushing stylet 825 causes catheter 822 to move superiorly, within right atrium 76. For some such applications, stylet 825 (e.g., supporting portion 828 and distal end 830 thereof) also moves superiorly, such that the atrial height 831b is greater than atrial height d831 shown in FIG. 12B. It is hypothesized by the inventors that pushing force 844 facilitates retracting distal housing 864 through prosthetic valve 868.

Typically, after retracting distal housing 864 through prosthetic valve 868, delivery tool 820 is withdrawn proximally from right atrium 76, via inferior vena cava 72.

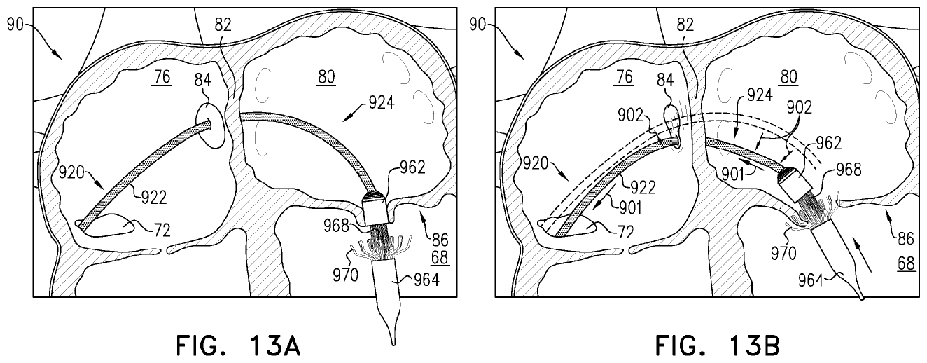

Reference is now made to FIGS. 13A-B, and 14A-D, which are schematic illustrations showing transfemoral-transseptal access to the left atrium of a subject, either with or without facilitation with a stylet 925, in accordance with some applications of the invention. FIGS. 13A-B illustrate an undesirable effect that may come about during transfemoral-transseptal implantation of some prosthetic mitral valves, and FIGS. 14A-D illustrate how the use of a stylet 925, in accordance with some applications of the invention, may diminish this undesirable effect.

FIG. 13A shows a delivery tool 920, comprising a steerable catheter 922, having been used to transfemorally-transseptally deliver a prosthetic valve 968 within a housing (e.g., comprising a proximal housing 962 and a distal housing 964) of the delivery tool, and at least ventricular snares 970 of the prosthetic valve having been deployed from the delivery tool (e.g., from distal housing 964) within left ventricle 68 of the heart. For some applications, delivery tool 920 is similar to other delivery tools described hereinabove, mutatis mutandis. For some applications, prosthetic valve 968 is similar to other prosthetic valves described hereinabove, mutatis mutandis.

FIG. 13B shows prosthetic valve 968 having been moved proximally, e.g., by pulling one or more components of tool 920 (such as catheter 922) proximally, in order to engage snares 970 with tissue of the mitral valve. Because of the curvature of the transfemoral-transseptal path to the mitral valve, and therefore the curvature of tool 920, the proximal pulling used to move prosthetic valve 968 proximally (represented by arrows 901) results in the tool repositioning and/or reshaping to take a shorter path to mitral valve 86 (represented by arrows 902). (The position of tool 920 in FIG. 13A is shown in phantom in FIG. 13B.)

One or more undesirable results may occur as a result of this behavior. For example, rather than prosthetic valve 968 moving in a predominantly upstream direction (e.g., along an atrioventricular axis), the upstream portion of the prosthetic valve may tilt toward fossa ovalis 84, thereby placing the prosthetic valve in a suboptimal orientation with respect to the mitral valve (e.g., as shown in FIG. 13B). Furthermore, as the portion of catheter 922 that is disposed within the heart moves toward the apex of the heart, it may exert an undesirable force (e.g., a shear force) on fossa ovalis 84, which may stretch or tear the fossa ovalis, (e.g., as shown in FIG. 13B).

FIGS. 14A-D shows use of a delivery tool 920′, comprising catheter 922′ to perform similar steps, but facilitated by the use of stylet 925. Delivery tool 920′ is typically identical to delivery tool 920, with the exception that catheter 922′ defines a secondary lumen 927 through which stylet 925 is advanceable.

For some applications, elements of delivery tool 920′ (e.g., catheter 922′ and stylet 925) are packaged together commercially, e.g., in a kit.

For some applications, stylet 925 is similar to other stylets described hereinabove, mutatis mutandis.

FIG. 14A shows the same state of implantation as FIG. 13A. Stylet 925 is then advanced distally within secondary lumen 927 of catheter 922′ at least as far as right atrium 76, and typically until it reaches a vicinity (e.g., within 1 cm, such as within 0.5 cm) of fossa ovalis 84 (FIG. 14B). Alternatively, stylet 925 may be already disposed within catheter 922′ prior to advancement of the catheter. As described hereinabove, mutatis mutandis, the presence of stylet 925 increases the rigidity of the portion of catheter 922′ within which the stylet is disposed. This therefore typically straightens that portion of catheter 922, e.g., as illustrated by the transition between FIG. 14A and 14B. For some applications, and/or in some subjects (e.g., due to anatomy), this results in the catheter being positioned higher in fossa ovalis 84 (i.e., further away from the apex of the heart), e.g., as illustrated by the transition between FIG. 14A and 14B.

Catheter 922′ is subsequently pulled proximally in order to engage snares 970 with tissue of the mitral valve (FIG. 14C). For some applications, and as shown in FIG. 14C, as catheter 922′ (and secondary lumen 927 thereof) is pulled proximally, it may slide over stylet 925, which, as shown, may remain stationary with respect to the heart. Stylet 925 may be used to apply a supporting force to catheter 922, thereby preventing the pulling 901 of catheter 922′ from repositioning and/or reshaping of the catheter, and thereby reduces the likelihood of the undesirable results described hereinabove. For example, and as shown in FIG. 14C, compared to the position of delivery tool 920 in the absence of stylet 925, prosthetic valve 968 is less tilted toward fossa ovalis 84, and the fossa ovalis is less likely to be stretched or torn (or stretching or tearing is reduced). To further illustrate this, a phantom image of catheter 922, in its position in FIG. 13B, is shown in FIG. 14C for comparison.

As shown in FIG. 14D, subsequent to full deployment of prosthetic valve 968, stylet 925 may also facilitate withdrawal of delivery tool 920′ (e.g., distal housing 964). For example, and as shown in FIG. 14D, the presence of stylet 925 may allow housing 964 to be withdrawn axially through the center of prosthetic valve 968, with less or no tilt.

For some applications, and as shown, secondary lumen 927 does not reach the distal end of catheter 922′. Similarly to catheter 922, catheter 922′ comprises a steerable distal portion 924. For some applications, and as shown, secondary lumen 927 does not reach steerable distal portion 924, such that stylet 925 is advanceable, within the secondary lumen, to a point proximal of the steerable distal portion. It is hypothesized by the inventors that stylet 925 being advanceable only as far as a point proximal of steerable distal portion 924 may have a desirable effect of maintaining steerability of catheter 922′ (e.g., of steerable distal portion 924 thereof) while stylet 925 is advanced within the catheter. For example, the limited length of secondary lumen 927 may prevent the operator from inadvertently advancing stylet 925 into steerable distal portion 924 and thereby inadvertently inhibiting steerability.

Alternatively, secondary lumen 927 may reach the steerable distal portion 924 and/or the distal end of catheter 922′. It is hypothesized by the inventors that this may facilitate withdrawal, to a greater degree, of catheter 922′ out of left atrium 80 while retaining stylet 925 in place.

It is to be noted that the techniques described with reference to FIGS. 14A-D may alternatively be performed, mutatis mutandis, by using a support other than a stylet, such as support 40, support 140, support 240, support 340, support 440, or support 540, described hereinabove.

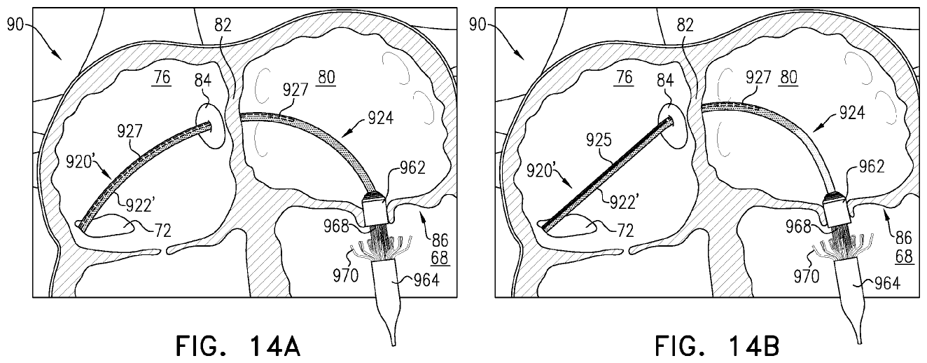

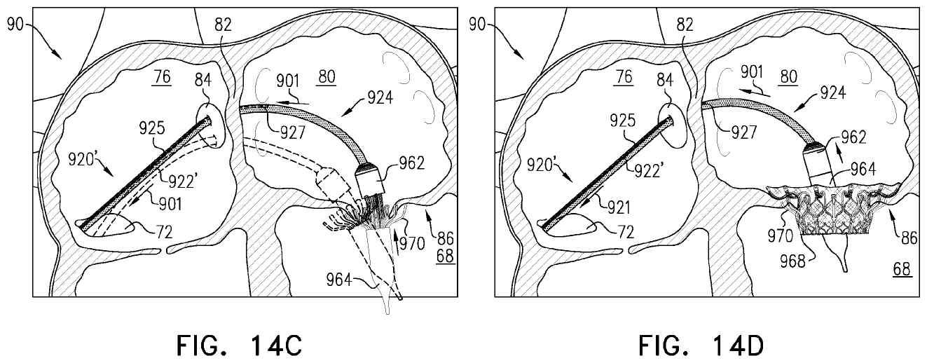

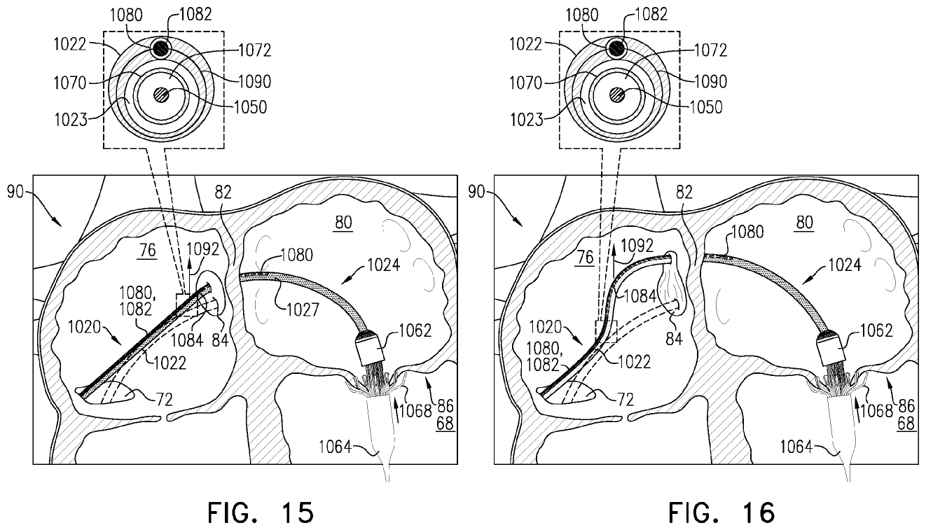

Reference is made to FIGS. 15-16, which are schematic illustrations showing use of a delivery tool 1020, comprising a catheter 1022 and a pull-wire 1082, for transfemorally implanting a prosthetic valve 1068 at a native valve 86 of the heart, in accordance with some applications of the present invention.

For some applications, elements of delivery tool 1020 (e.g., catheter 1022 and pull-wire 1082) are packaged together commercially, e.g., in a kit.

Delivery tool 1020 is similar in certain aspects to delivery tool 820. Components that are identically named between the systems typically share similar features and serve similar functions as each other. For example, delivery tool 1020 comprises catheter 1022 that defines a primary lumen 1023 through which a shaft 1070 passes, similarly to shaft 870 that passes through primary lumen 823 of catheter 822. Further similarly to delivery tool 820, guidewire 1050 passes through a shaft-lumen 1072 defined by shaft 1070, and a secondary lumen 1080 is defined by catheter 1022.

Typically, and in contrast to delivery tool 820, delivery tool 1020 comprises a pull-wire 1082 that passes through a pull-wire lumen 1080 that is defined by catheter 1022 (similarly to secondary lumen 827 of catheter 822, mutatis mutandis). As shown in the respective insets of FIGS. 15-16, delivery tool 1020 further comprises a pull-ring 1090 that is fastened (e.g., soldered) to pull-wire 1082. For some applications, and as shown, pull-ring 1090 is embedded within a wall of catheter 1022. Typically for such applications, pull-wire 1082 extends proximally from pull-ring 1090 to a proximal portion (e.g., at a proximal portion of delivery tool 1020), such that a pulling force, when applied to the proximal portion of the pull-wire, is transferred, via pull-ring 1090, to a portion of catheter 1022, as described hereinbelow.

FIGS. 15-16 show catheter 1022 having been advanced to the stage in delivery of prosthetic valve 1068 from housings 1062, 1064 described hereinabove with reference to FIG. 14C. Pulling the proximal portion of pull-wire 1082 changes an arc of catheter 1022 within right atrium 76, as shown by comparison to the phantom image of catheter 922 in FIG. 13B.

In FIG. 15, pull-ring 1090 is embedded within a portion of catheter 1022 that is adjacent to fossa ovalis 84, such that a tensioned portion 1084 that is located distal of the pull ring and abutting the fossa ovalis is elevated (arrow 1092).

It is hypothesized by the inventors that so using pull-wire 1082 to tension portion 1084 of catheter 1022 may reduce shear forces being applied to fossa ovalis 84, reducing the risk of cheesewiring described hereinabove.

In FIG. 16, pull-ring 1090 is embedded within a portion of catheter 1022 that is further proximal from fossa ovalis 84 than as in FIG. 15. Thus, pulling on pull-wire 1082 elevates a tensioned portion 1084 that is longer than as shown in FIG. 15. For some applications, and as shown, portions of catheter 1022 that are desired to become tensioned portions 1084 (i.e., portions of catheter that are distal of pull-ring 1090, yet proximal of steerable distal portion 1024) are more flexible (e.g., are thinner) than portions of catheter that are proximal of the pull-ring. It is hypothesized by the inventors that (i) tensioned portion 1084 comprising material having a greater flexibility than material comprising proximal portions of catheter, and/or (ii) pull-ring 1090 being disposed at a greater distance from fossa ovalis 84, facilitate increased elevation of tensioned portion 1084 upon pulling pull-wire 1082 (FIG. 16).

For some applications, in addition to pull-wire lumen 1080, catheter 1022 further defines a secondary lumen through which a stylet may be advanced, as described hereinabove in reference to FIGS. 14A-D, mutatis mutandis.

The apparatus and techniques described in the present patent application may be applied to those described in one or more of the following patent application publications. For example, the delivery tools, catheters, and prosthetic valves described in the present application may represent, or may be substituted with, one or more of the delivery tools, catheters, and prosthetic valves described in one or more of the following patent application publications, each of which is incorporated herein by reference in its entirety:
US 2015/0157457 to HaCohen
US 2013/0172992 to Gross et al.
US 2014/0324164 to Gross et al.
US 2014/0257475 to Gross et al.
US 2014/0207231 to HaCohen et al.
US 2015/0351906 to Hammer et al.
US 2017/0266003 to Hammer et al.
US 2018/0014930 to Hariton et al.
US 2018/0280136 to Hariton et al.
US 2017/0231766 to Hariton et al.
US 2019/0231525 to Hariton et al.
US 2019/0167423 to Hariton et al.
WO 2019/030753 to Hariton et al.
WO 2019/026059 to Hariton et al.
WO 2019/116369 to Hariton et al.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a kit, the kit comprising:
a flexible catheter, the catheter being transfemorally advanceable to a heart of a subject; and
a support, the support being dimensioned for percutaneous access to the heart, the support defining:
a proximal end,
an elongate portion, and
a distal portion having a catheter-engaging element, the catheter-engaging element being configured to reversibly engage the catheter in a manner that does not inhibit longitudinal advancement of the catheter.

2. The apparatus according to claim 1, wherein the catheter and the support are configured such that, while the catheter-engaging element engages the catheter, pulling on the proximal end of the support applies a superiorly-directed force to the catheter.

3. The apparatus according to claim 1, wherein the catheter-engaging element is shaped to form a loop, the loop being dimensioned to facilitate advancing the catheter through the loop.

4. The apparatus according to claim 3, wherein the loop is tightenable around the catheter when the loop is engaged with the catheter.

5. The apparatus according to claim 4, further comprising a tightening ring, the loop being threaded through the tightening ring, such that advancing the tightening ring, along the loop, tightens the loop around the catheter.

6. The apparatus according to any one of claims 1-3, wherein the catheter-engaging element is transitionable in shape from a non-engaging state into an engaging state.

7. The apparatus according to claim 6, further comprising a pull-wire attached to the catheter-engaging element, wherein the catheter-engaging element is configured to be bent, using the pull-wire, from the non-engaging state into the engaging state.

8. The apparatus according to claim 6, further comprising a constraint, wherein the catheter-engaging element comprises a shape-memory material, such that:
the catheter-engaging element is constrainable in the non-engaging state by the constraint, and
upon removal of the constraint, the catheter-engaging element automatically transitions into the engaging state.

9. The apparatus according to claim 8, wherein the constraint comprises a constraining overtube, and wherein the catheter-engaging element is disposed within the constraining overtube.

10. The apparatus according to claim 8, wherein the constraint comprises a stiff internal rod disposed within the catheter-engaging element.

11. The apparatus according to any one of claims 1-3, wherein the catheter-engaging element comprises a snare, the snare configured to engage the catheter through an opening of the snare.

12. The apparatus according to claim 11, wherein the opening of the snare has a width that is greater than an external diameter of the catheter.

13. The apparatus according to any one of claims 1-3, wherein:
the catheter comprises a support-engaging element coupled to an outer wall of the catheter, and
the catheter-engaging element is configured to engage the support-engaging element.

14. The apparatus according to claim 13, wherein:
the support-engaging element is shaped to define a rail extending along a portion of the catheter,
the catheter-engaging element is shaped to define a hook, and
the hook is configured to ensnare the rail.

15. The apparatus according to claim 13, wherein:
the support-engaging element comprises a ferromagnetic material,
the catheter-engaging element comprises a ferromagnetic material, and
the catheter-engaging element is configured to magnetically engage the support-engaging element.

16. The apparatus according to claim 15, wherein the support-engaging element comprises an electromagnet.

17. The apparatus according to claim 15, wherein the catheter-engaging element comprises an electromagnet.

18. Apparatus comprising a kit, the kit comprising:
a flexible catheter, the catheter:
being transfemorally advanceable to a heart of a subject, and
defining a primary lumen and a secondary lumen therethrough;
a prosthetic valve, the prosthetic valve being dimensioned to be passed to the heart through the primary lumen; and
a stylet that is stiffer than the flexible catheter, and is slidable through the secondary lumen.

19. The apparatus according to claim 18, wherein the stylet is rigid.

20. The apparatus according to claim 18, wherein the kit further comprises a piercing device, the piercing device being configured to pierce an interatrial septum of the heart.

21. The apparatus according to any one of claims 18-20, wherein the kit further comprises a dilator, the dilator being configured to dilate a pierced interatrial septum of the heart.

22. A method for use at a heart of a subject, the method comprising:
advancing a distal end of a catheter transfemorally and through an interatrial septum of the heart into a left side of the heart; and
while a distal end of a stylet is disposed inside the catheter and within a right atrium of the heart, withdrawing the distal end of the catheter proximally toward the interatrial septum such that the catheter slides over the stylet.

23. The method according to claim 22, further comprising, subsequently to the step of advancing the distal end of the catheter, and prior to the step of withdrawing the distal end of the catheter, advancing the distal end of the stylet through the catheter such that the distal end of the catheter becomes disposed within the right atrium of the heart, inside the catheter.

24. The method according to claim 22, wherein the step of advancing the distal end of the catheter comprises advancing the distal end of the catheter transfemorally and through the interatrial septum of the heart into the left side of the heart while the distal end of the stylet is disposed inside the catheter.

25. The method according to claim 22, further comprising, subsequently to withdrawing the distal end of the catheter proximally toward the interatrial septum, withdrawing the distal end of the catheter from the heart.

26. The method according to any one of claims 22-25, wherein a prosthetic valve is coupled to the catheter, and wherein the step of advancing the distal end of the catheter comprises advancing the distal end of the catheter transfemorally and through the interatrial septum of the heart into the left side of the heart such that the prosthetic valve becomes advanced transfemorally and through the interatrial septum of the heart into the left side of the heart.

27. The method according to claim 26, further comprising implanting the prosthetic valve at a mitral valve of the heart.

28. A method for use at a heart of a subject, the method comprising:
advancing a distal portion of a catheter, via an inferior vena cava of the subject, to a right atrium of the heart, through an interatrial septum of the heart and into a left atrium of the heart;
advancing a support, via a superior vena cava of the subject, to the right atrium of the heart;
engaging the catheter with the support within the right atrium of the heart; and
while supporting the catheter in the right atrium using the support, withdrawing the distal end of the catheter proximally toward the interatrial septum.

29. A method for transseptally accessing a left atrium of a heart of a subject, the method comprising:
advancing a catheter, via an inferior vena cava of the subject, to a right atrium of the heart;
advancing a support, via a superior vena cava of the subject, to the right atrium of the heart;
advancing a distal portion of the catheter through an interatrial septum of the heart and into the left atrium;
engaging the catheter with the support within the right atrium of the heart; and
subsequently, steering the distal portion of the catheter toward a mitral valve of the heart, while supporting the catheter in the right atrium using the support.

30. The method according to claim 29, wherein advancing the catheter to the right atrium comprises advancing the catheter to the right atrium subsequently to advancing the support to the right atrium.

31. The method according to claim 29, wherein advancing the catheter to the right atrium comprises advancing the catheter to the right atrium prior to advancing the support to the right atrium of the heart.

32. The method according to any one of claims 29-31, wherein the support includes a catheter-engaging element, and engaging the catheter with the support comprises engaging the catheter with the catheter-engaging element.

33. The method according to claim 32, wherein the catheter includes a support-engaging element, and engaging the catheter with the catheter-engaging element comprises engaging the support-engaging element to the catheter-engaging element.

34. The method according to claim 33, wherein engaging the support-engaging element to the catheter-engaging element comprises magnetically engaging the support-engaging element to the catheter-engaging element.

35. The method according to claim 34, wherein the support-engaging element includes an electromagnet, and magnetically engaging the support-engaging element to the catheter-engaging element comprises activating the electromagnet.

36. The method according to claim 34, wherein the catheter-engaging element includes an electromagnet, and magnetically engaging the support-engaging element to the catheter-engaging element comprises activating the electromagnet.

37. The method according to claim 36, wherein:
the support-engaging element is a first support-engaging element,
magnetically engaging the support-engaging element to the catheter-engaging element comprises magnetically engaging the first support-engaging element to the catheter-engaging element,
the catheter includes at least a second support-engaging element, and
the method further comprises, subsequently to magnetically engaging the first support-engaging element to the catheter-engaging element:
ceasing the magnetic engagement between the first support-engaging element and the catheter-engaging element by deactivating the electromagnet; and
subsequently, magnetically engaging the second support-engaging element to the catheter-engaging element by reactivating the electromagnet.

38. The method according to claim 32, wherein engaging the catheter with the catheter-engaging element comprises mechanically engaging the catheter with the catheter-engaging element.

39. The method according to claim 38, wherein mechanically engaging the catheter with the catheter-engaging element comprises fitting a protrusion into a recess.

40. The method according to claim 38, wherein:
the catheter includes a support-engaging element shaped to define a rail,
the catheter-engaging element is shaped to define a hook, and
engaging the catheter with the catheter-engaging element comprises hooking the rail with the hook.

41. The method according to claim 38, wherein the catheter-engaging element is shaped to form a loop, and mechanically engaging the catheter with the catheter-engaging element comprises advancing the distal portion of the catheter through the loop of the catheter-engaging element.

42. The method according to claim 38, wherein the catheter-engaging element is shaped to define a snare, and mechanically engaging the catheter with the catheter-engaging element further comprises supporting a supported portion of the catheter with the snare.

43. The method according to claim 42, wherein supporting the supported portion comprises supporting the supported portion while the supported portion is disposed within the right atrium.

44. The method according to claim 42, wherein the catheter-engaging element is shaped to form a loop, and supporting the supported portion with the catheter-engaging element comprises tightening the loop around the catheter.

45. The method according to claim 38, wherein the catheter-engaging element has a non-engaging state and an engaging state, and advancing the support to the right atrium comprises advancing the support while the catheter-engaging element is in the non-engaging state.

46. The method according to claim 45, wherein engaging the catheter comprises transitioning the catheter-engaging element from the non-engaging state to the engaging state.

47. The method according to any one of claims 29-46, wherein supporting the catheter in the right atrium using the support comprises, subsequently to advancing the distal portion of the catheter through the interatrial septum, moving the catheter by applying a force to the catheter, using the support.

48. The method according to claim 47, wherein applying the force to the catheter comprises applying a supporting force to the catheter using the support by applying tension to the support.

49. The method according to claim 47, wherein moving the catheter using the support comprises using the support to change an orientation of the catheter.

50. The method according to claim 47, wherein supporting the catheter further comprises, subsequently to moving the catheter by applying the force to the catheter, continuing to apply the force to the catheter.

51. The method according to claim 50, wherein applying the force to the support facilitates steering the distal portion of the catheter toward the mitral valve.

52. The method according to claim 47, wherein moving the catheter comprises moving a supported portion of the catheter, relative to the interatrial septum.

53. The method according to claim 52, wherein moving the supported portion of the catheter relative to the interatrial septum comprises moving the supported portion of the catheter towards the superior vena cava.

54. The method according to any one of claims 29-53, wherein the distal portion of the catheter includes a piercing device, and advancing the distal portion of the catheter through the interatrial septum comprises piercing the septum with the piercing device.

55. The method according to claim 54, wherein the piercing device includes a needle, and piercing the septum comprises mechanically puncturing the septum with the needle.

56. The method according to any one of claims 29-55, wherein:
advancing the catheter, via the inferior vena cava, to the right atrium comprises advancing an implant within the catheter, via the inferior vena cava, to the right atrium; and
the method further comprises implanting the implant at the mitral valve.

57. The method according to claim 56, wherein the implant includes a prosthetic heart valve, wherein implanting the implant at the mitral valve comprises implanting the prosthetic heart valve at the mitral valve.

58. The method according to claim 56, wherein the implant includes an annuloplasty device, wherein implanting the implant at the mitral valve comprises implanting the annuloplasty device at the mitral valve.

59. A method for transfemorally implanting a prosthetic valve at a native valve of a heart of a subject, the method comprising:
advancing a delivery tool, via an inferior vena cava of the subject, to a right atrium of the heart;
advancing a support, via a superior vena cava of the subject, to the right atrium of the heart;
engaging the delivery tool with the support within the right atrium of the heart;
steering a distal portion of the delivery tool toward the native valve, while supporting the delivery tool in the right atrium using the support; and
subsequently, at the native valve, deploying a prosthetic valve from the delivery tool.

60. The method according to claim 59, wherein the support includes a catheter-engaging element having a non-engaging state and the engaging state, and engaging the delivery tool with the support within the right atrium of the heart comprises transitioning the catheter-engaging element from the non-engaging state to the engaging state.

61. The method according to claim 59, wherein:
steering the distal portion of the delivery tool toward the native valve comprises steering the distal portion of the delivery tool toward a tricuspid valve of the heart; and
deploying the prosthetic valve from the delivery tool comprises deploying the prosthetic valve from the delivery tool at the tricuspid valve.

62. The method according to any one of claims 59-61, further comprising, subsequently to deploying the prosthetic valve from the delivery tool:
withdrawing the support from the right atrium, via the superior vena cava; and
withdrawing the delivery tool from the right atrium, via the inferior vena cava.

63. The method according to claim 62, wherein:
the delivery tool includes a proximal housing and a distal housing,
deploying the prosthetic valve from the delivery tool comprises increasing a distance between the proximal housing and the distal housing, and
withdrawing the delivery tool proximally via the inferior vena cava comprises retracting the distal housing through the prosthetic valve.

64. The method according to claim 62, further comprising, subsequently to deploying the prosthetic valve from the delivery tool, disengaging the delivery tool from the support.

65. The method according to claim 64, wherein the support includes a catheter-engaging element having a non-engaging state and the engaging state, and disengaging the delivery tool from the support comprises transitioning the catheter-engaging element from the engaging state to the non-engaging state.

66. The method according to any one of claims 59-65, wherein supporting the delivery tool in the right atrium using the support comprises applying a force to the delivery tool using the support.

67. The method according to claim 66, wherein applying the force to the delivery tool using the support comprises moving the delivery tool using the support, by applying the force.

68. The method according to claim 67, wherein moving the delivery tool comprises changing an orientation of the distal portion of the delivery tool.

69. The method according to claim 67, wherein applying the force to the delivery tool using the support comprises applying the force to the delivery tool using the support while the prosthetic valve is at least partially disposed within a right ventricle of the heart.

70. The method according to claim 67, wherein applying the force to the delivery tool using the support comprises applying the force to the delivery tool using the support while the prosthetic valve is at least partially deployed from the delivery tool.

71. The method according to claim 67, wherein applying the force to the delivery tool using the support comprises coupling the prosthetic valve to a ventricular tissue of the heart.

72. The method according to claim 67, wherein applying the force to the delivery tool using the support comprises applying a superiorly-directed force to the delivery tool using the support.

73. The method according to claim 72, wherein applying the superiorly-directed force to the delivery tool using the support comprises applying the superiorly-directed force to the delivery tool using the support, while deploying the prosthetic valve from the delivery tool.

74. The method according to claim 72, wherein applying the superiorly-directed force to the delivery tool using the support comprises, subsequently to deploying the prosthetic valve, applying the superiorly-directed force to the delivery tool using the support while withdrawing at least a portion of the delivery tool into the right atrium.

75. The method according to claim 74, wherein applying the superiorly-directed force to the delivery tool using the support while withdrawing at least the portion of the delivery tool into the right atrium comprises applying the superiorly-directed force to the delivery tool using the support while withdrawing the distal housing into the right atrium.

76. A method for transseptally accessing a left atrium of a heart of a subject, the method comprising:
transluminally positioning a delivery tool comprising a catheter and a stylet such that:
the catheter extends, via an inferior vena cava of the subject, to a right atrium of the heart, and
the stylet extends, within the catheter, to the right atrium; and
while a distal end of the stylet remains in the right atrium:
steering a distal portion of the catheter away from the distal end of the stylet and through an interatrial septum of the heart into the left atrium; and
subsequently, steering the distal portion of the catheter toward a mitral valve of the heart.

77. The method according to claim 76, wherein extending the catheter, via the inferior vena cava, to the right atrium, comprises extending the catheter, via the inferior vena cava, to the right atrium, while the catheter is empty of the stylet.

78. The method according to claim 76, wherein extending the catheter, via the inferior vena cava, to the right atrium, comprises extending the catheter, via the inferior vena cava, to the right atrium, while the stylet is disposed within the catheter.

79. The method according to claim 76, wherein the catheter defines a secondary lumen therethrough, and extending the stylet within the catheter and to the right atrium comprises extending the stylet through the secondary lumen.

80. The method according to any one of claims 76-79, wherein the catheter houses an implant, and the method further comprises:
advancing the implant within the catheter, via the inferior vena cava, to the right atrium; and
subsequently to steering the distal portion of the catheter toward the mitral valve, deploying the implant from within catheter, at the mitral valve.

81. The method according to claim 80, wherein the implant includes a prosthetic heart valve, and implanting the implant at the mitral valve comprises implanting the prosthetic heart valve at the mitral valve.

82. The method according to claim 80, wherein the implant includes an annuloplasty device, wherein implanting the implant at the mitral valve comprises implanting the annuloplasty device at the mitral valve.

83. The method according to any one of claims 76-82, wherein the delivery tool further includes a piercing device, the piercing device extending distally out of the distal end of catheter, and wherein steering the distal end of the catheter through the interatrial septum comprises piercing the septum with the piercing device.

84. The method according to claim 83, wherein the piercing device includes a needle, and piercing the septum comprises mechanically puncturing the septum with the needle.

85. The method according to any one of claims 76-84, wherein steering the distal portion of the catheter away from the distal end of the stylet and through the interatrial septum comprises steering the distal portion of the catheter away from the distal end of the stylet and through the interatrial septum while the stylet extends within the right atrium, to an atrial height that is between 1 and 70 mm.

86. The method according to claim 85, wherein steering the distal portion of the catheter away from the distal end of the stylet and through the interatrial septum while the stylet extends within the right atrium, to an atrial height that is between 1 and 70 mm comprises steering the distal portion of the catheter away from the distal end of the stylet and through the interatrial septum while the stylet extends within the right atrium, to an atrial height that is between 1 and 10 mm.

87. The method according to claim 85, wherein steering the distal portion of the catheter away from the distal end of the stylet and through the interatrial septum while the stylet extends within the right atrium, to an atrial height that is between 1 and 70 mm comprises steering the distal portion of the catheter away from the distal end of the stylet and through the interatrial septum while the stylet extends within the right atrium, to an atrial height that is between 10 and 20 mm.

88. The method according to claim 85, wherein steering the distal portion of the catheter away from the distal end of the stylet and through the interatrial septum while the stylet extends within the right atrium, to an atrial height that is between 1 and 70 mm comprises steering the distal portion of the catheter away from the distal end of the stylet and through the interatrial septum while the stylet extends within the right atrium, to an atrial height that is between 10 and 70 mm.

89. The method according to claim 88, wherein steering the distal portion of the catheter away from the distal end of the stylet and through the interatrial septum while the stylet extends within the right atrium, to an atrial height that is between 10 and 70 mm comprises steering the distal portion of the catheter away from the distal end of the stylet and through the interatrial septum while the stylet extends within the right atrium, to an atrial height that is between 20 and 70 mm.

90. The method according to claim 89, wherein steering the distal portion of the catheter away from the distal end of the stylet and through the interatrial septum while the stylet extends within the right atrium, to an atrial height that is between 20 and 70 mm comprises steering the distal portion of the catheter away from the distal end of the stylet and through the interatrial septum while the stylet extends within the right atrium, to an atrial height that is between 20 and 30 mm.

91. The method according to claim 89, wherein steering the distal portion of the catheter away from the distal end of the stylet and through the interatrial septum while the stylet extends within the right atrium, to an atrial height that is between 20 and 70 mm comprises steering the distal portion of the catheter away from the distal end of the stylet and through the interatrial septum while the stylet extends within the right atrium, to an atrial height that is between 30 and 60 mm.

92. The method according to claim 89, wherein steering the distal portion of the catheter away from the distal end of the stylet and through the interatrial septum while the stylet extends within the right atrium, to an atrial height that is between 20 and 70 mm comprises steering the distal portion of the catheter away from the distal end of the stylet and through the interatrial septum while the stylet extends within the right atrium, to an atrial height that is between 40 and 70 mm.

93. The method according to any one of claims 76-92, wherein steering the distal portion of the catheter toward the mitral valve, while the distal end of the stylet remains in the right atrium, comprises steering the distal portion of the catheter toward the mitral valve, while the stylet extends within the right atrium, to an atrial height that is between 1 and 70 mm.

94. The method according to claim 93, wherein steering the distal portion of the catheter toward the mitral valve, while the stylet extends within the right atrium, to an atrial height that is between 1 and 70 mm, comprises steering the distal portion of the catheter toward the mitral valve, while the stylet extends within the right atrium, to an atrial height that is between 1 and 10 mm.

95. The method according to claim 93, wherein steering the distal portion of the catheter toward the mitral valve, while the stylet extends within the right atrium, to an atrial height that is between 1 and 70 mm, comprises steering the distal portion of the catheter toward the mitral valve, while the stylet extends within the right atrium, to an atrial height that is between 10 and 20 mm.

96. The method according to claim 93, wherein steering the distal portion of the catheter toward the mitral valve, while the stylet extends within the right atrium, to an atrial height that is between 1 and 70 mm, comprises steering the distal portion of the catheter toward the mitral valve, while the stylet extends within the right atrium, to an atrial height that is between 10 and 70 mm.

97. The method according to claim 96, wherein steering the distal portion of the catheter toward the mitral valve, while the stylet extends within the right atrium, to an atrial height that is between 10 and 70 mm, comprises steering the distal portion of the catheter toward the mitral valve, while the stylet extends within the right atrium, to an atrial height that is between 20 and 70 mm.

98. The method according to claim 97, wherein steering the distal portion of the catheter toward the mitral valve, while the stylet extends within the right atrium, to an atrial height that is between 20 and 70 mm, comprises steering the distal portion of the catheter toward the mitral valve, while the stylet extends within the right atrium, to an atrial height that is between 20 and 30 mm.

99. The method according to claim 97, wherein steering the distal portion of the catheter toward the mitral valve, while the stylet extends within the right atrium, to an atrial height that is between 20 and 70 mm, comprises steering the distal portion of the catheter toward the mitral valve, while the stylet extends within the right atrium, to an atrial height that is between 30 and 60 mm.

100. The method according to claim 97, wherein steering the distal portion of the catheter toward the mitral valve, while the stylet extends within the right atrium, to an atrial height that is between 20 and 70 mm, comprises steering the distal portion of the catheter toward the mitral valve, while the stylet extends within the right atrium, to an atrial height that is between 40 and 70 mm.

101. A method for transfemorally accessing a native valve of a heart of a subject, the method comprising:
positioning a delivery tool comprising a catheter and a stylet, such that:
the catheter extends, via an inferior vena cava of the subject, to a right atrium of the heart, and
the stylet extends, within the catheter, to the right atrium; and
while a distal end of the stylet remains in the right atrium, and within the catheter:
steering a distal portion of the delivery tool away from the distal end of the stylet and toward the native valve.

102. The method according to claim 101, further comprising, subsequently to the step of steering, and while the distal end of the stylet remains in the right atrium, and within the catheter:
deploying a prosthetic valve from the delivery tool, at the native valve.

103. The method according to claim 101, wherein the native valve is a tricuspid valve of the heart.

104. The method according to claim 101, wherein:
the native valve is a mitral valve of the heart, and
the step of steering comprises advancing the distal portion of the delivery tool through an interatrial septum of the heart into a left atrium of the heart.

105. The method according to claim 101, wherein positioning the delivery tool comprises advancing the delivery tool transluminally via the inferior vena cava.

106. The method according to claim 101, wherein the catheter defines a secondary lumen therethrough, and extending the stylet within the catheter and to the right atrium comprises extending the stylet through the secondary lumen.

107. The method according to claim 106, wherein positioning the delivery tool comprises positioning the delivery tool such that the stylet extends, within the catheter and through a secondary lumen, to the right atrium.

108. The method according to any one of claims 101-107, wherein steering the distal portion of the delivery tool away from the distal end of the stylet and toward the native valve comprises sliding the catheter distally over the stylet.

109. The method according to claim 108, wherein sliding the catheter distally over the stylet comprises holding the distal end of the stylet at a generally constant atrial height while steering the distal portion toward and through the native valve.

110. The method according to claim 102, wherein deploying the prosthetic valve from the distal portion of the delivery tool comprises extracorporeally applying a proximally-directed force to the delivery tool.

111. The method according to claim 110, wherein extracorporeally applying the proximally-directed force to the delivery tool comprises extracorporeally applying the proximally-directed force to the delivery tool such that the prosthetic valve moves proximally and engages tissue of the native valve.

112. The method according to claim 110, wherein the delivery tool includes a distal housing, and extracorporeally applying the proximally-directed force to the delivery tool comprises increasing a distance between a distal opening of the catheter and the distal housing.

113. The method according to claim 110, wherein extracorporeally applying the proximally-directed force to the delivery tool comprises retracting the delivery tool through the prosthetic valve.

114. The method according to claim 110, wherein extracorporeally applying the proximally-directed force to the delivery tool comprises holding the distal end of the stylet in the right atrium, and within the catheter, a generally constant atrial height, by applying the proximally-directed force to catheter, and applying a pushing force to the stylet.

115. The method according to any one of claims 101-114, wherein steering the distal portion of the delivery tool away from the distal end of the stylet and toward the native valve, while the distal end of the stylet remains in the right atrium, and within the catheter, comprises steering the distal portion of the catheter away from the distal end of the stylet and toward the native valve, while the stylet extends within the right atrium, to an atrial height that is between 1 and 70 mm.

116. The method according to claim 115, wherein steering the distal portion of the catheter away from the distal end of the stylet and toward the native valve, while the stylet extends within the right atrium, to an atrial height that is between 1 and 70 mm comprises steering the distal portion of the catheter away from the distal end of the stylet and toward the native valve, while the stylet extends within the right atrium, to an atrial height that is between 1 and 10 mm.

117. The method according to claim 115, wherein steering the distal portion of the catheter away from the distal end of the stylet and toward the native valve, while the stylet extends within the right atrium, to an atrial height that is between 1 and 70 mm comprises steering the distal portion of the catheter away from the distal end of the stylet and toward the native valve, while the stylet extends within the right atrium, to an atrial height that is between 10 and 20 mm.

118. The method according to claim 115, wherein steering the distal portion of the catheter away from the distal end of the stylet and toward the native valve, while the stylet extends within the right atrium, to an atrial height that is between 1 and 70 mm comprises steering the distal portion of the catheter away from the distal end of the stylet and toward the native valve, while the stylet extends within the right atrium, to an atrial height that is between 10 and 70 mm.

119. The method according to claim 118, wherein steering the distal portion of the catheter away from the distal end of the stylet and toward the native valve, while the stylet extends within the right atrium, to an atrial height that is between 10 and 70 mm comprises steering the distal portion of the catheter away from the distal end of the stylet and toward the native valve, while the stylet extends within the right atrium, to an atrial height that is between 20 and 70 mm.

120. The method according to claim 119, wherein steering the distal portion of the catheter away from the distal end of the stylet and toward the native valve, while the stylet extends within the right atrium, to an atrial height that is between 20 and 70 mm comprises steering the distal portion of the catheter away from the distal end of the stylet and toward the native valve, while the stylet extends within the right atrium, to an atrial height that is between 20 and 30 mm.

121. The method according to claim 119, wherein steering the distal portion of the catheter away from the distal end of the stylet and toward the native valve, while the stylet extends within the right atrium, to an atrial height that is between 20 and 70 mm comprises steering the distal portion of the catheter away from the distal end of the stylet and toward the native valve, while the stylet extends within the right atrium, to an atrial height that is between 30 and 60 mm.

122. The method according to claim 119, wherein steering the distal portion of the catheter away from the distal end of the stylet and toward the native valve, while the stylet extends within the right atrium, to an atrial height that is between 20 and 70 mm comprises steering the distal portion of the catheter away from the distal end of the stylet and toward the native valve, while the stylet extends within the right atrium, to an atrial height that is between 40 and 70 mm.

123. The method according to claim 102, wherein deploying the prosthetic valve from the delivery tool, while the distal end of the stylet remains in the right atrium, and within the catheter, comprises deploying the prosthetic valve from the delivery tool while the stylet extends within the right atrium, to an atrial height that is between 1 and 70 mm.

124. The method according to claim 123, wherein deploying the prosthetic valve from the delivery tool while the stylet extends within the right atrium, to an atrial height that is between 1 and 70 mm, comprises deploying the prosthetic valve from the delivery tool while the stylet extends within the right atrium, to an atrial height that is between 1 and 10 mm.

125. The method according to claim 123, wherein deploying the prosthetic valve from the delivery tool while the stylet extends within the right atrium, to an atrial height that is between 1 and 70 mm, comprises deploying the prosthetic valve from the delivery tool while the stylet extends within the right atrium, to an atrial height that is between 10 and 20 mm.

126. The method according to claim 123, wherein deploying the prosthetic valve from the delivery tool while the stylet extends within the right atrium, to an atrial height that is between 1 and 70 mm comprises deploying the prosthetic valve from the delivery tool while the stylet extends within the right atrium, to an atrial height that is between 10 and 70 mm.

127. The method according to claim 126, wherein deploying the prosthetic valve from the delivery tool while the stylet extends within the right atrium, to an atrial height that is between 10 and 70 mm comprises deploying the prosthetic valve from the delivery tool while the stylet extends within the right atrium, to an atrial height that is between 20 and 70 mm.

128. The method according to claim 127, wherein deploying the prosthetic valve from the delivery tool while the stylet extends within the right atrium, to an atrial height that is between 20 and 70 mm comprises deploying the prosthetic valve from the delivery tool while the stylet extends within the right atrium, to an atrial height that is between 20 and 30 mm.

129. The method according to claim 128, wherein deploying the prosthetic valve from the delivery tool while the stylet extends within the right atrium, to an atrial height that is between 20 and 70 mm comprises deploying the prosthetic valve from the delivery tool while the stylet extends within the right atrium, to an atrial height that is between 30 and 60 mm.

130. The method according to claim 128, wherein deploying the prosthetic valve from the delivery tool while the stylet extends within the right atrium, to an atrial height that is between 20 and 70 mm comprises deploying the prosthetic valve from the delivery tool while the stylet extends within the right atrium, to an atrial height that is between 40 and 70 mm.

131. A method for transseptally accessing a mitral valve of a heart of a subject, the method comprising:
transluminally positioning a catheter such that the catheter extends, via an inferior vena cava of the subject, to a right atrium of the heart;
advancing a distal portion of the catheter through an interatrial septum of the heart into a left atrium of the heart;
subsequently, tilting the distal portion of the catheter toward the mitral valve by transluminally extending a stylet, within the catheter, to the right atrium.

132. The method according to claim 131, wherein transluminally extending the stylet, within the catheter, to the right atrium, comprises extracorporeally pushing a proximal portion of the stylet towards a superior vena cava of the heart.

133. The method according to claim 131, wherein tilting the distal portion of the catheter comprises tilting the distal portion of the catheter such that the distal opening of the catheter faces the mitral valve.

134. The method according to any one of claims 131-133, wherein, while a distal end of the stylet remains in the right atrium, steering the distal portion of the catheter toward a mitral valve of the heart.

135. The method according to claim 134, wherein the catheter houses an implant, and the method further comprises, subsequently to tilting the distal portion of the catheter, deploying the implant from within the catheter, at the mitral valve.

136. The method according to claim 135, wherein the implant includes a prosthetic heart valve, and implanting the implant at the mitral valve comprises implanting the prosthetic heart valve at the mitral valve.

137. The method according to claim 135, wherein the implant includes an annuloplasty device, wherein implanting the implant at the mitral valve comprises implanting the annuloplasty device at the mitral valve.

138. The method according to any one of claims 131-137, wherein transluminally extending the stylet, within the catheter, to the right atrium, comprises extending the stylet, within the right atrium, to an atrial height between 1 and 70 mm.

139. The method according to claim 138, wherein extending the stylet, within the right atrium, to an atrial height between 1 and 70 mm, comprises extending the stylet to an atrial height between 1 and 10 mm.

140. The method according to claim 138, wherein extending the stylet, within the right atrium, to an atrial height between 1 and 70 mm, comprises extending the stylet to an atrial height between 10 and 20 mm.

141. The method according to claim 138, extending the stylet, within the right atrium, to an atrial height between 1 and 70 mm, comprises extending the stylet to an atrial height between 10 and 70 mm.

142. The method according to claim 141, wherein extending the stylet, within the right atrium, to an atrial height between 10 and 70 mm, comprises extending the stylet to an atrial height between 20 and 70 mm.

143. The method according to claim 142, wherein extending the stylet to an atrial height between 20 and 70 mm, comprises extending the stylet to an atrial height between 20 and 30 mm.

144. The method according to claim 142, wherein extending the stylet to an atrial height between 20 and 70 mm, comprises extending the stylet to an atrial height between 30 and 60 mm.

145. The method according to claim 142, wherein extending the stylet to an atrial height between 20 and 70 mm, comprises extending the stylet to an atrial height between 40 and 70 mm.
